(19) United States
(12) Patent Application Publication
(10) Pub. No.: US 2024/0153588 A1
(43) Pub. Date: May 9, 2024

(54) SYSTEMS AND METHODS FOR IDENTIFYING MICROBIAL BIOSYNTHETIC GENETIC CLUSTERS

(71) Applicant: PRAGMA BIOSCIENCES INC., San Carlos, CA (US)

(72) Inventors: Kareem BARGHOUTI, San Carlos, CA (US); Ayin VALA, San Carlos, CA (US); Kovi BESSOFF, San Carlos, CA (US); Peter MCCAFFREY, San Carlos, CA (US)

(21) Appl. No.: 18/549,972

(22) PCT Filed: Mar. 11, 2022
(86) PCT No.: PCT/US2022/071098

§ 371 Date: Sep. 11, 2023

Related U.S. Application Data
(60) Provisional application No. 63/160,655, filed on Mar. 12, 2021.

(51) Int. Cl. G16B 30/10; G16B 40/20; G16H 20/10; G16H 50/70

(52) U.S. Cl. CPC G16B 30/10; G16B 40/20; G16H 20/10; G16H 50/70

(57) ABSTRACT

Embodiments of the disclosure include systems, methods, and compositions related to identification of biomarkers and drug candidates from the gut microbiome based on analysis of Biosynthetic Gene Clusters (BGCs) from bacteria in the microbiome. The systems are generated with ranking of the BGCs using novel artificial intelligence overlayed with information about patient response to immune checkpoint immunotherapy. The disclosed platform allows for identification of a response outcome from an individual in need of immune checkpoint immunotherapy.

This application claims priority to U.S. Provisional Patent Application 63/160,655, filed Mar. 12, 2021, which is incorporated by reference herein in its entirety.

### BACKGROUND

### Technical Field

Embodiments of this disclosure relate at least to the fields of medicine, including cancer medicine, bioinformatics, and artificial intelligence.

### Background

Cancer therapy is in a state of disruptive transition. Where historically the field has used broadly toxic chemotherapy drugs or extremely specific targeted therapies, the field now turns to the immune system as a robust and precise tool for finding and eradicating the disease. The revolution of immunotherapy has brought hope to formerly untreatable diseases through the stimulation and direction of the host immune cells to fight cancer. However, despite the significant breakthroughs of drugs in recent years, guiding the immune system is a complex process requiring the mutual effort of immune stimulation and direction. Microbes have evolved a powerful capacity to do this and those that live within the human gut demonstrate a uniquely evolved capability to modulate the immune systems of their hosts. By focusing on these microbes and the compounds they produce, we can source novel molecular therapies by harnessing eons of evolutionary drug design culminating within and around us.

### BRIEF SUMMARY

Embodiments of the disclosure encompass systems, methods, and compositions related to identification of compounds, including metabolites, that can act as biomarkers, as therapies themselves, and/or as therapeutic leads for cancer therapy, including at least checkpoint inhibition therapy. In specific embodiments, the disclosure also provides the produced systems, methods, and compositions for use for an individual in need of immune checkpoint immunotherapy (ICI) and that has an unknown response to ICI.

In some cases, the disclosure provides systems, methods, and compositions for identifying a response outcome for an individual that will receive ICI of any kind. Examples of ICI may include anti-PD1 drugs (e.g., Pembrolizumab, Nivolumab, and Cemiplimab), anti-PD-L1 drugs (e.g., Atezolizumab, Avelumab, Durvalumab), and anti-CTLA-4 drugs (e.g., Ipilimumab). In particular embodiments, the present disclosure addresses the need in the art to identify how a patient will respond to an immune checkpoint therapy, given that individuals have different microbiome profiles.

In particular embodiments, the systems, methods, and compositions provide a platform comprised of two general processes: a first process that utilizes sequence information from bacteria in stool samples from patients having a known response to ICI and that identifies a group of BGCs related to the known responses, and a second that utilizes AI to rank the BGCs in order of their statistical association of the responses to ICI.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates one embodiment of the disclosure in which Biosynthetic Gene Clusters (BGCs) are grouped according to similarity and then further narrowed to target BGCs through consideration of therapy response information.

FIG. 2 illustrates one embodiment of a pipeline of the disclosure starting from obtaining patient stool samples, through BGC analysis and annotation and AI ranking of the BGCs for relevance.

FIG. 3 shows an example of a schematic workflow for the extraction, processing and quality control of stool samples.

FIG. 4 demonstrates a plot illustrating the distribution of % GC content compared between published microbiome data (401) and data sequenced using Applicant's pipeline, Patient Cohort 1 (403) and Patient Cohort 2 (404) depict microbial DNA exclusively, yellow (402) depicts contaminant human DNA removed from Applicant's samples).

FIG. 5 illustrates an example of a gene mining workflow prior to (optional) taxonomic profiling and BGC extraction steps, in accordance with various embodiments.

FIG. 6 illustrates an example of a gene mining workflow that happens before (optional) taxonomic profiling and BGC extraction steps, in accordance with various embodiments.

FIG. 7 demonstrates an example of a schematic workflow illustrating the biostatistical assessment of a sample cohort using the PERMANOV A method, in accordance with various embodiments.

FIGS. 8A and 8B show a visual representation of a BGC similarity network. In FIG. 8A, circles represent BGCs (nodes) that are color-coded based on metadata components. Lines (edges) connect these nodes that exceed a minimum threshold of similarity to form families.

FIG. 8B provides one example of the relationship between nodes, edges, and families.

FIG. 9 illustrates an example of a schematic workflow illustrating a direct graph-based method for identification of target BGC families, in accordance with various embodiments.

FIG. 10 shows an example of a schematic workflow illustrating topological data analysis process, in accordance with various embodiments.

FIG. 11 demonstrates an example schematic workflow illustrating a machine learning workflow, in accordance with various embodiments.

FIG. 12 provides a plot of the performance of an applicant model for predicting outcomes in ICI therapy, in accordance with various embodiments.

FIG. 13 illustrates a computer system that may be implemented in embodiments of the disclosure, in accordance with various embodiments.

FIG. 14 provides an example implementation of the platform of the disclosure applied to a new set of patients having unknown response to ICI therapy, in accordance with various embodiments.

FIG. 15 provides plots illustrating that BGCs identified by the platform of the disclosure are able to stratify patients response in several cohorts, in accordance with various embodiments.

FIG. 16 illustrates an example workflow for training an artificial intelligence (AI) model for assessing or predicting clinical outcomes in immunotherapy patients using identified microbial biosynthetic genetic cluster (BGCs), in accordance with various embodiments.

FIG. 17 illustrates an example workflow for assessing or predicting clinical outcomes in immunotherapy patients using identified microbial biosynthetic genetic cluster (BGCs), in accordance with various embodiments.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DETAILED DESCRIPTION

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The system, compositions, and methods for their use can “comprise,” “consist essentially of,” or “consist of” any of the elements or steps disclosed throughout the specification. Compositions and methods “consisting essentially of” any of the elements or steps disclosed limits the scope of the claim to the specified materials or steps that do not materially affect the basic and novel characteristic of the claimed invention.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

It is contemplated that any embodiment discussed in this specification can be implemented with respect to any method or composition of the disclosure, and vice versa. Furthermore, compositions of the disclosure can be used to achieve methods of the disclosure.

Immune checkpoint immunotherapy (ICI) is a revolutionary treatment strategy for many advanced for formerly intractable cancer diagnoses. This revolutionary potential is driven in large part by the fact that these therapies disinhibit the host immune system to then identify and attack cancerous cells. Unfortunately, patient response rates to these medications are widely variable with some indications having response rates below 20% (Darvin et al. 2018). The investigation into both predictive biomarkers and adjuvant factors for response had been ongoing for the past several years with findings demonstrating that the gut microbiome is composed of different taxonomic groups in ICI responders versus non-responders and that fecal transplantation or inoculation of key taxa from responders into murine models of melanoma improves anti-PD-1 and anti-CTLA-4 ICI response prospectively (Gopalakrishnan et al. 2017; Vetizou et al. 2015; Matson et al. 2018; Routy et al. 2017).

Metagenomics is the study of genetic material retrieved directly from environmental samples rather than single organisms. Currently, whole-genome shotgun sequencing (WGS) is employed to achieve a broad recovery of genomic material across all organisms in an ecosystem sample. Shotgun metagenomics empowers microbial ecology to be investigated in far greater detail than earlier microbial genome sequencing methods such as 16s marker gene sequencing.

A large and diverse set of enzymatic pathways that produce specialized metabolites in bacteria, fungi, and plants are known to be encoded in Biosynthetic Gene Clusters (BGCs). Living organisms produce a range of secondary metabolites and many of these secondary metabolites can be used as natural products in medicine. It is possible to computationally identify BGCs in genome sequences and to systematically explore and prioritize them for their role in clinical outcomes. Retrieving BGCs from metagenome samples can be successful using, for example, the application of whole-genome shotgun sequencing followed by the assembly of short sequencing reads into longer, contiguous genomic sequences that can contain the whole, identifiable BGCs.

The various embodiments herein introduce the methods to produce immunomodulatory and significant metagenomic Biosynthetic Gene Clusters as well as a catalog of these BGCs that can be utilized to predict response for individuals on checkpoint therapy, which also can be used for the development of drugs.

The term “cancer” refers to a large collection of heterogeneous genetic disorders with drastically different clinical manifestations and prognoses. Traditional therapeutics have been non-specific (e.g. traditional chemotherapy), resulting in a high frequency of adverse effects, while the use of more recently developed therapies directed at specific tumor mutations (e.g. epidermal growth factor receptor (EGFR) mutations in lung cancer) is limited by the small population sizes that meet the requirements associated with these indications. While considerable progress has been made, curative therapies have yet to be discovered for many devastating oncology diagnoses, as evidenced by a recent study by Lu et al. (2017) that identified over 140 oncologic indications with “unmet medical needs”. In addition to underscoring the urgent need for novel therapies, this designation provides substantial incentives (including breakthrough status) for the pharmaceutical industry to develop new therapeutic candidates, as these programs may be eligible for special consideration by the FDA under its Expedited Programs designations.

The normal human immune system relies on a complex system of signaling molecules to maintain normal function (a process often referred to as homeostasis). One major class of signaling molecules, known as immune checkpoint proteins, plays an important role in preventing the immune response from becoming overactive that would result in autoimmune disease (this control process is known as immune tolerance). However, this same signaling axis has been shown to be co-opted by cancer cells to allow them to evade immune detection.

Previous related work predominantly describes the microbiome as a composition of microbial taxa, typically using either 16S marker gene sequencing or whole genome shotgun to assign taxonomic labels and relative abundances to metagenome sequencing data. Clinical applications of these approaches, therefore, seek to associate particular taxa with clinical outcome. As such, many studies produced by different research groups disagree about which specific taxa are important. This disagreement between studies is largely because of bioinformatic limitations inherent to the approach of taxonomic labeling, namely, that utilization of marker genes—whether limited to the 16S region of bacterial genomes or inclusive of broader genomic information in the case of whole-genome shotgun sequencing—ultimately results in the selection of the nearest match in a reference database of whole-genome sequences. Simply generating whole-genome shotgun sequencing data does not circumvent this limitation because it is a difference of analytical approach whether this data is used to identify nearest whole-genome references from a database or to identify BGCs.

The various embodiments described herein can employ whole-genome shotgun sequencing to directly examine the genetic content of the entire metagenome. While more precise, this method has traditionally been prohibitively expensive but, more recently, is achievable with increasing scale due to new, higher-throughput sequencers.

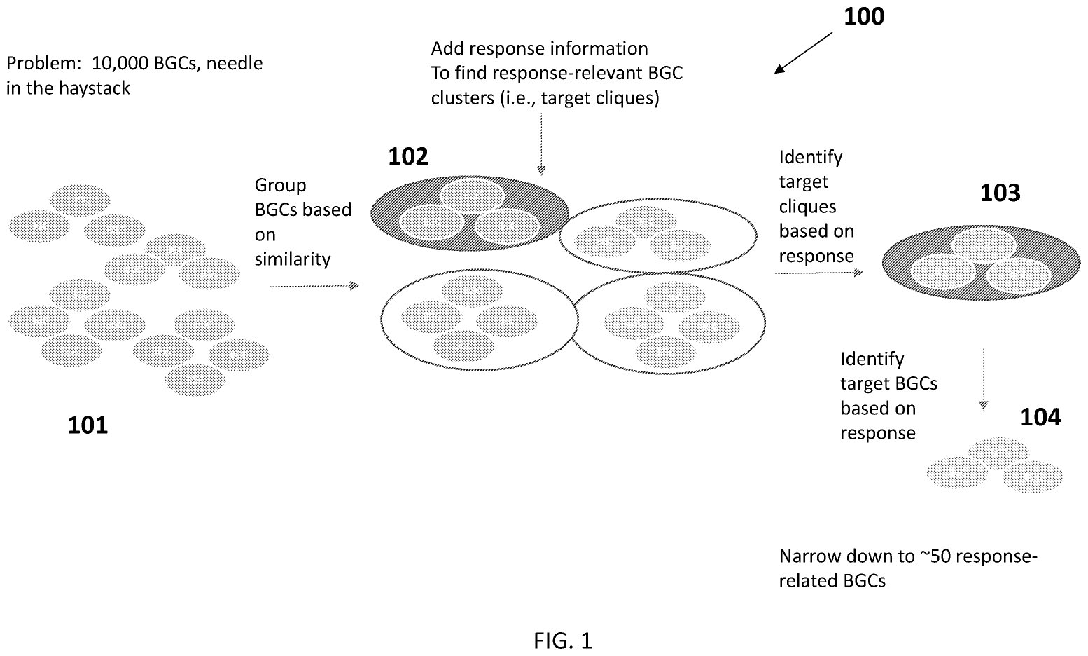

As discussed herein, the Biosynthetic Gene Clusters (BGCs) space is very large, and hence identifying key BGCs is difficult. In accordance with various embodiments herein, methods are provided to make the high dimensional space into a smaller space by grouping BGCs. Response information can then be overlayed providing a better feature space for artificial intelligence (AI) to identify key response-relevant BGCs. The example workflow provided in FIG. 1 illustrates a general, non-limiting workflow 100 to solve this problem. FIG. 1 illustrates a collection of BGCs that are group based on similarity 101. Once grouped, response information can be added to identify response-relevant BGC clusters 102 (illustrated by the shaded cluster 102), such as target cliques identified based on response. Those target cliques can be further interrogated to identify target BGCs based on response 103. As a result, a sample method is provided that can take a cohort of ˜10,000 BGCs and narrow that cohort down to a specific subgroup 104 (such as ˜50 BGCs) associated with a specific response.

### A Genetic Mining Pipeline to Produce Biomarkers and Therapeutic Leads for Checkpoint Inhibition Therapy

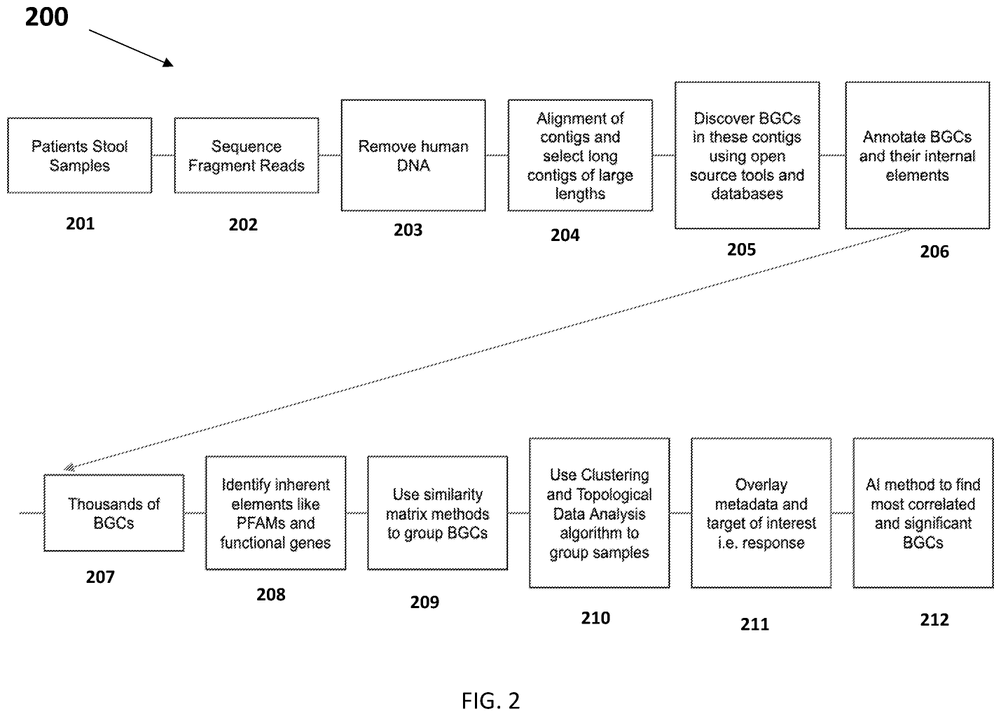

The present disclosure concerns a computational and molecular technology platform that collects, organizes, and processes sequencing data to identify, compare, prioritize, and describe Biosynthetic Gene Clusters (BGCs) and their products. Embodiments of the disclosure encompass a grouping and ranking algorithm that selects gene clusters most correlated with patient response to immunotherapy. This allows for a rational, systematic approach to map the chemical space of the human gut microbiome and is a useful catalyst for drug and biomarker discovery. FIG. 2 provides a schematic illustration of an example workflow 200 for the genetic mining pipeline, in accordance with various embodiments. Generally, stool samples from patients are obtained 201 and nucleic acid in the sample is sequenced to produce fragment reads 202. Host human DNA is removed from the sample 203, and the sequence reads are aligned as larger contigs 204. The contigs are analyzed for identification of BGCs utilizing open source tools and databases 205. Following this, information from a database allows annotation of BGCs and their internal elements 206, such as core biosynthetic genes and their protein families, transport related genes, regularity genes, resistance gens, etc. for thousands of BGCs 207. Analysis and annotation of the BGCs identifies regions of interest that may be further annotated with gene and protein family (PFAM) labels 208. The BGCs are grouped utilizing similarity matrix methods 209. BGCs are grouped upon utilizing a clustering and topological data analysis algorithm 210. Metadata is overlayed with this grouping for response information 211, and an AI method identifies the statistically significant BGCs 212.

Using whole metagenome shotgun sequencing, novel immunoregulatory gene clusters were identified through the de novo assembly of short-read (e.g., approximately 150 bp) metagenome sequencing data generated from patient stool samples. Contigs assembled from these short-read data sets are then annotated for gene content, metabolic function, and probable structure as a Biosynthetic Gene Cluster (BGC) according to the computational pipeline. The identified gene clusters are grouped and ranked according to the clinical response status of the patient from whom they were collected. This results in groups of homologous gene clusters ranked according to their correlation with host response to checkpoint blockade immunotherapy. In particular embodiments, from these rankings, one can determine immunoregulatory function.

### General Molecular Embodiments

Embodiments of the disclosure provide a platform that identified BGCs and their corresponding gene product(s) that are correlative with the ability of an individual to effectively respond to an ICI. The accrued information in the platform also allows for its use to apply to an individual having an unknown response to the ICI. Production of the platform generally begins with sequencing of gut microbiome bacterial DNA (although in alternative cases the sample comes from blood) to the exclusion of the host DNA and in some cases DNA from ingested material from organisms, where the host is an individual that has received ICI and has a known response to it, such as complete response, partial response, stable disease, progressive disease, etc. In specific embodiments, the sequencing is whole genome shotgun sequencing. The purpose of sequencing allows for identification of multiple BGCs in the bacterial DNA, and in particular embodiments shorter sequencing reads are collated into larger contiguous sequences that are no less than about 20 kb in length. The upper limit of these contigs may be in the metabase range. The lower limit for the length of the contigs raises the likelihood that an entire BGC will be captured on one contig. A BGC may have 2, 3, 4, 5, or more genes that together encode a biosynthetic pathway for the production of a secondary metabolite (a product synthesized by the bacteria that is not crucial for its growth). In some embodiments, one or more BGCs are already known prior to sequencing from a patient. In certain embodiments, one or more gene products of the BGC is unknown and/or has not been annotated.

Once the sequencing is complete and the contigs are obtained, the sequence of the respective BGCs is analyzed for similarity with other BGCs such that they are then grouped based on ICI therapy response outcome (which may or may not include different ICI therapies). For example, the BGCs may be grouped based on those associated with responders to the therapy. This response-targeted group of BGCs may then be utilized in an algorithm that ranks them based on statistical response to the clinical therapy. This allows for identification of target cliques based on response that may be applied to an individual with an unknown response to ICI.

In specific embodiments, the presently disclosed system may not utilize taxonomic information of the bacteria as part of the bioinformatics component of the system. However, upon identification of one or more relevant BGCs, the taxonomy of the BGCs may be known and may be consulted. In alternative embodiments, taxonomic information of the bacteria may be used as part of the bioinformatics component of the system.

Embodiments of the disclosure include systems comprising information suitable for obtaining clinical information for an individual in need of a clinical therapy, such as information for the likelihood of efficacy for at least one therapy for the individual. The system may encompass information of stored patient data, including response to one or more therapies, gender, age, race, and so forth. The system may comprise information, including ranked information, that is based on success or lack thereof for one or more therapies. The ranked information may be encompass a spectrum of types of responses to therapy, and such ranking may be searchable. In specific embodiments, the system may comprise information related to sequencing data, such as sequence of microbial genomic DNA that is sourced from the gut of individuals having a known response to a therapy. In particular embodiments, the system has stored information of rankings of BGCs from bacteria based on whether or not the host of the bacteria responded to a therapy, and the stored information is of a searchable form. In particular embodiments, the system may apply a greater value for BGCs from gut bacteria in which the host responded to the therapy than BGCs from gut bacteria in which the host did not respond to the therapy. In certain embodiments, within the rankings of the system there may be certain BGCs even among responder hosts that may have a higher statistical association with response to therapy than others. In specific embodiments, the system may also include gene annotation, taxa, and gene clusters associated with one or a group of BGCs.

Embodiments of the disclosure may include systems comprising information that may be utilized for development of therapeutic compounds of any kind. In particular embodiments, the system may include information related to BGCs from gut microbes in which the host previously was administered a therapy. The therapeutic compound may or may not be developed for the same purpose as the therapy received by the host individual. In specific embodiments, the system may comprise information related to BGCs ranked based on efficacy of the therapy, and the information (particularly for those that responded) can be employed for identification of one or more therapeutic candidates. In specific embodiments, the system can encompass information about metabolite(s) producible from higher ranked BGCs, and therefore associated with efficacy of a clinical response. This information from the system may be utilized for employment of the metabolite(s) itself as the therapy or as a lead compound for development of other structurally and/or functionally related compounds. Any therapeutic candidate or lead compound may be tested in vitro or in vivo for efficacy, such as having the same activity as the therapy that the host received. In some cases, in silico analysis may be pursued to obtain additional information of a therapeutic candidate or lead compound. In specific cases, the system may provide information about therapeutic candidates or lead compounds themselves.

Part or all of any system encompassed herein may or may not be web-based, such as for transmittal of patient sequence, transmittal of database sequence, transmittal of clinical information, patient data of any kind, a combination thereof, and so forth.

Use of WGS Instead of 16s

The two most common approaches to sequencing the content of the microbiome (referred to as the metagenome) are marker gene sequencing and whole genome shotgun (WGS) sequencing, and in particular embodiments the systems and methods of the disclosure may utilize WGS. Marker gene sequencing takes advantage of the fact that bacterial genomes contain areas that are conserved enough to be captured by general sequencing primers but variable enough—in theory—to be unique to specific organisms. The most popular example of marker gene sequencing is known as 16S ribosomal sequencing and is a cornerstone of many microbiome-based publications and even therapeutic development programs. This approach is advantageous in that it provides a sort of molecular fingerprint for each of the hundreds of bacteria in the gut in an efficient and cost-effective manner. However, marker gene sequencing only directly captures a very small portion of each bacterium's genome, and fills in the rest by using that small portion to search among a database of reference genomes. This means that marker gene sequencing is limited in its precision and is relatively insensitive to genetic transposition and strain-level evolution. On the contrary, WGS sequencing directly examines the genetic content of the entire metagenome. While more precise, this method has traditionally been prohibitively expensive but, more recently, is achievable with increasing scale due to new, higher-throughput sequencers.

Microbial DNA Extractions from Stool

Pursuing WGS sequencing and obtaining a comprehensive and direct picture of the metagenome not only allows for more accurate taxonomic resolution but also allows for the extraction of other important features apart from the taxonomy (e.g., Biosynthetic Gene Clusters). To allow this, one may extract DNA in a way to produce longer contigs (such as, no less than 20 Kb). In addition, it may be useful to minimize batch effects while processing hundreds of samples.

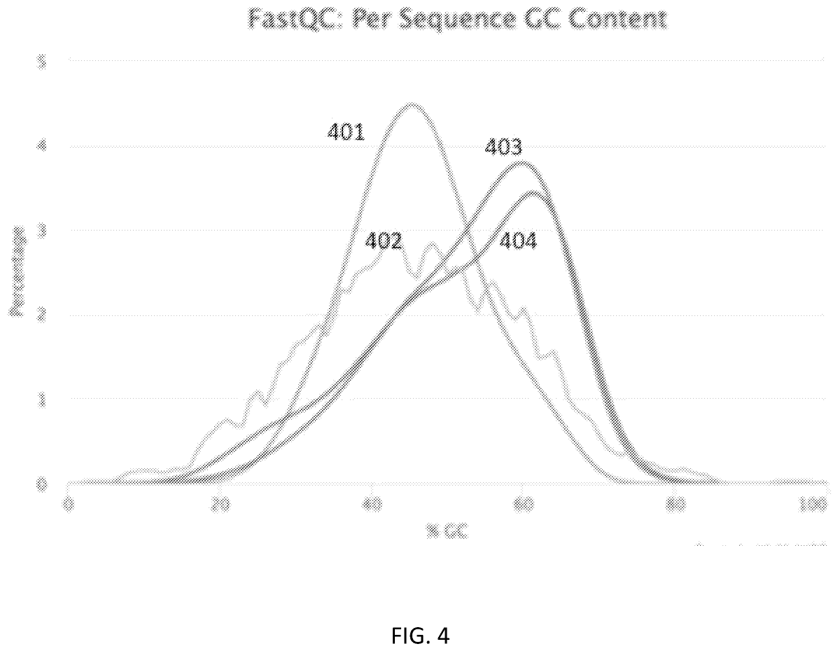

The sequencing pipeline for the disclosure may be optimized to increase sequencing coverage of difficult to sequencing regions of the bacterial genome. The sequencing of a large clinical cohort (n=331 metagenomes) using a customized Illumina pipeline incorporating the Nextera FLEX and NovaSeq6000 has been completed. It has been demonstrated that the approach generates longer contigs and increased coverage of GC-rich genomes, a useful functionality for gut metagenome sequencing in various embodiments (see plots and data provided by FIG. 4). FIG. 4A provides a plot illustrating the distribution of % GC content compared between published microbiome data (401) and data sequenced using Applicant's pipeline (403 and 404 depict microbial DNA exclusively, 402 depicts contaminant human DNA removed from Applicant's samples).

### Sample Processing and QC

Stool may undergo DNA extraction in batches with appropriate positive and negative controls. DNA yield can be quantified and recorded for quality control monitoring. Before the samples are processed, a subset of samples can be run as a pilot to ensure data quality. Raw read counts and sequence base quality plus length, as well as phix and human contamination levels, can be assessed for QC purposes before continuing to sequence the entire sample set. With each run of biological sample batches, mock community (positive) controls may also be run on each batch. American Type Culture Collection (ATCC®) mock communities MSA-1003 and MSA-2002 as examples may be included on each run. A single stool sample can also be run on each run consistently as a control, in various embodiments. Finally, a blank sample (negative control) may be included at the DNA extraction step (to control for contamination at the extraction step) and a second mock included in the library creation step (to control for contamination introduced at the library creation step). The quality pilot samples may be rerun in the main batches with the rest of the samples. These can be distributed across the main batches. In various embodiments, samples are scrambled across batches for the important outcome and clinical variables in the metadata to reduce the potential for “batch effects”.

A modified version of the QIAamp PowerfecalDNA Pro Extraction Kit protocol may be used for DNA extraction. Briefly, frozen stool may be thawed and aliquoted into a garnet bead tube with 60 uL of lysis buffer. Next, the lysate may be mechanically lysed using a bead beater (Qiagen TissueLyser) at 30 Hz for three minutes. The cell lysate, now containing both human and microbial DNA, can then be purified and isolated using the Qiagen Powersoil DNA Extraction Kit per manufacturer's instructions. A cDNA library may be synthesized, and samples can be index barcoded and pooled for high-throughput sequencing. Pre-library DNA extractions may be quantitated using the Nanodrop, in specific cases.

Library construction may be done for all samples simultaneously to minimize variability produced by batch effects and to maintain consistency across all samples. DNA fragment size analysis can be performed using the DNA Tapestation (Agilent) with a target insert size of 300-500 bp. mWGS sequencing libraries can be constructed using the Nextera DNA FLEX Library Preparation Kit (Illumina). MiSeq runs may be used as QC before full high throughput runs (DNA & RNA). The MiSeq run can confirm adequate sequence yield for the library, and adequate sample representation across the library (e.g. confirm the library is not just a single sample being represented).

Sequencing may be done using Illumina instruments to generate 2×150 bp paired-end reads at 40 million reads per sample using the NovaSeq 6000. Samples may be sequenced at depths greater than or equal to 5 GB. Duplicates and human contaminants may be filtered out, and adapters and low-quality bases can be trimmed. The resulting clean reads can be used for taxonomical and metabolic function analysis.

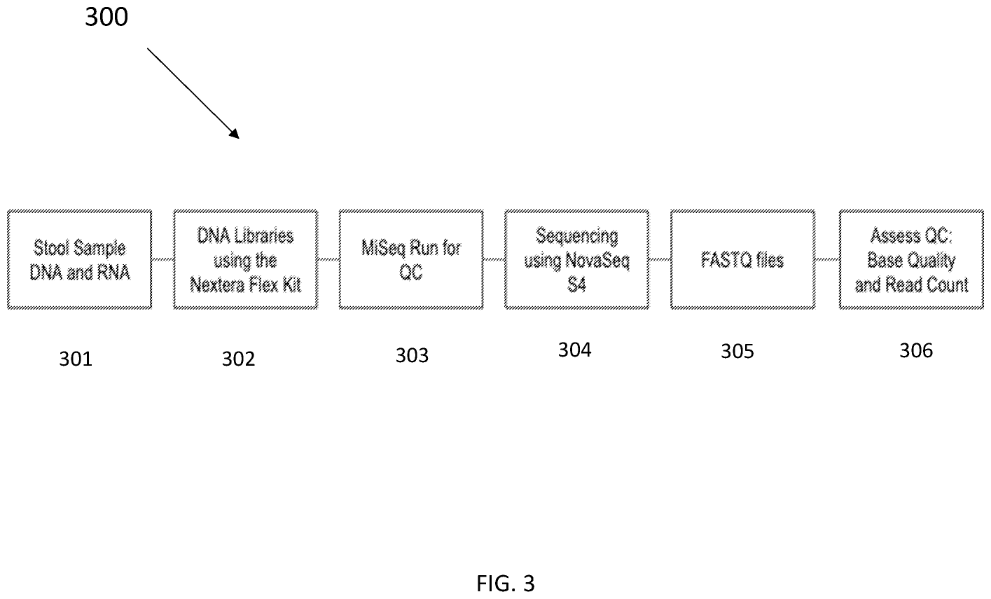

One example of Sample Processing and QC may be as follows (see FIG. 3 for example schematic workflow 300 for the extraction, processing and QC of stool samples, in accordance with various embodiments):
1. Stool sample DNA and RNA may be extracted from stool samples 301. DNA and RNA yield are quantified and reported for quality control monitoring, in various embodiments. BioAnalyzer traces, RNA integrity numbers (RINs), and DNA/RNA yields can be examined.
2. DNA libraries are prepared for shotgun metagenomic sequencing using the Nextera Flex kit 302, as one example.
3. MiSeq runs ma be used as QC before full high throughput runs (DNA & RNA) 303. The MiSeq run confirms adequate sequence yield for the library, and adequate sample representation across the library (e.g., confirm the library is not just a single sample being represented).
4. One may perform sequencing using the NovaSeq S4 platform 304 for 2×150 bp paired-end reads. Samples can be sequenced at depths greater than or equal to 5 gb for DNA (metagenomics) and greater than or equal to 40 million reads for RNA (meta-transcriptomics).
5. Raw sequencing data may be delivered in FASTQ format 305.
6. Before the samples are processed, a subset of samples can be run as a pilot to ensure data quality. Raw read counts and sequence base quality+length, as well as phix and human contamination levels, may be assessed for QC purposes 306 before continuing to sequence the entire sample set.
7. With each run of biological sample batches, mock community controls can also be run on each batch. American Type Culture Collection (ATCC®) mock communities MSA-1003 and MSA-2002 can be included on each run. A single stool sample is also run on each run consistently as a control, in certain cases. Finally, a blank sample may be included at the DNA extraction step (to control for contamination at the extraction step) and a second mock included in the library creation step (to control for contamination introduced at the library creation step).
8. The quality pilot samples may be rerun in the main batches with the rest of the samples. These are distributed across the main batches.
9. The samples can be processed at the end of the study for sequencing.
10. Samples are scrambled across batches for the important outcome and clinical variables in the metadata to reduce the potential for “batch effects”, in various embodiments

Gene Assembly

Improved sequencing inputs, when combined with de novo assembly tools, allow for improved recovery of primary metagenomic content (as opposed to reference genomes). This provides useful input data for subsequent analysis, reduces noise, and yields more robust predictions, in various embodiments.

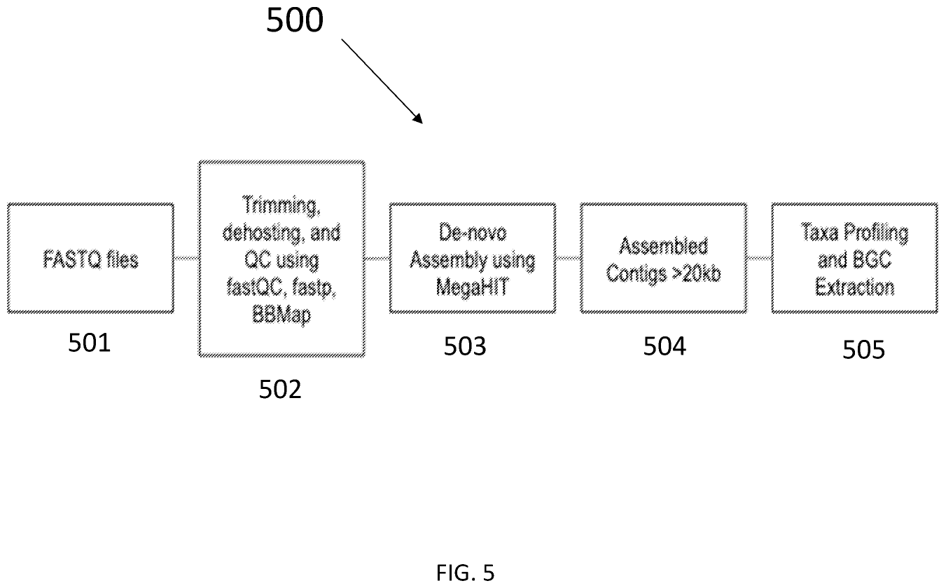

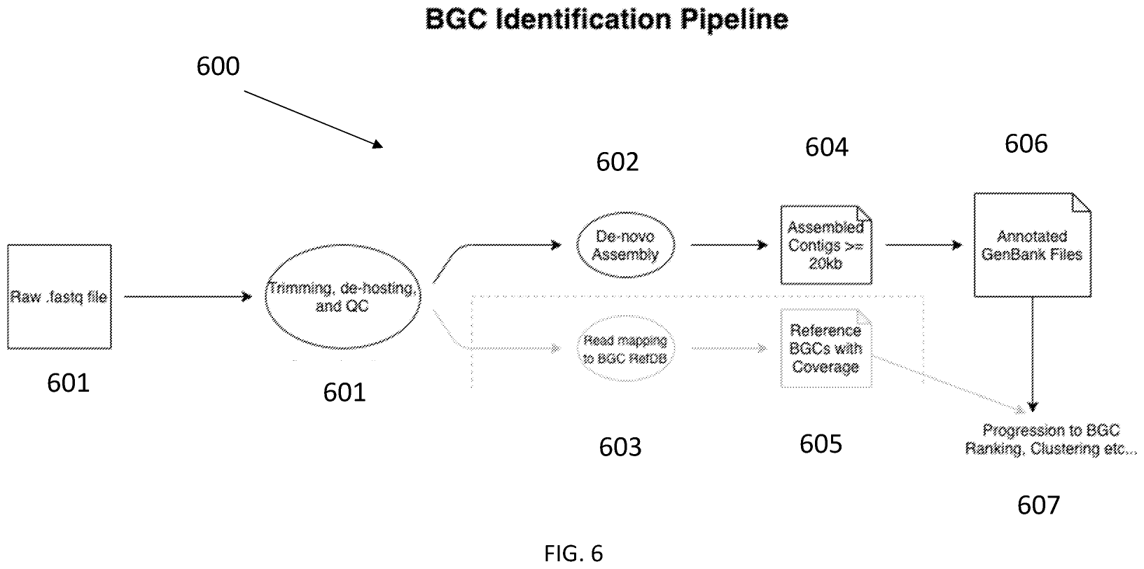

The present disclosure may employ an approach commonly and generally referred to as “gene mining” to identify BGCs. This process proceeds from DNA sequencing to genome assembly, BGC identification, BGC comparison, and ultimately BGC selection, in various embodiments. FIG. 5 provides an example gene mining workflow 500, preceding taxonomic profiling and BGC extraction steps (e.g., workflows) discussed below, in accordance with various embodiments. In workflow 500, FASTQ files 501 are subjected to trimming of extraneous sequences, removal of host DNA, and quality control using one or more methods 502. Utilization of MegaHIT results in de novo assembly of contigs 503, such as those that are greater than 20 kb in length 504. The contigs may then be subject to taxonomic profiling and BGC extraction 505. FIG. 6 also provides an example gene mining workflow 600, preceding taxonomic profiling and BGC extraction steps (e.g., workflows), in accordance with various embodiments. Raw sequencing data 601 is trimmed, host DNA sequences are removed, and the information is subject to quality control 601. In various embodiments, de novo assembly of contigs 602 results in assembly of contigs greater than 20 kb 604. The contigs are annotated for gene information 606, followed by BGC ranking and clustering 607. In an alternative embodiment, following trimming, etc., 601, there is read mapping to a reference database 603 that identifies BGCs with coverage, followed by BGC ranking and clustering 607.

As such, in various embodiments assembly is a useful aspect of effective BGC mining and requires that short sequencing reads (such as those produced by the NovaSeq 6000) be stitched together based upon overlapping ends to produce longer contiguous stretches of DNA called “contigs”. These contigs can then be systematically examined for regions likely to be BGCs, which produces a list of tens of thousands of BGCs representing the microbiota of an entire patient cohort. These BGCs and their associated clinical metadata may be further featurized and ranked in order of their statistical association to immunophenotype and clinical outcome. Subsequent sections explore each of these phases in greater detail.

The informatics pipeline seeks to align, annotate, and analyze metagenomic data producing BGCs for each patient and overlay clinically relevant metadata for further exploration. It includes the ingestion of raw FASTQ files produced by whole metagenome shotgun sequencing through QC, assembly, and annotation of genes, taxa, and gene clusters, in specific embodiments.

The accuracy of BGC prediction may be dependent upon the quality and the length contiguous genomic sequences produced by the assembly, known as “contigs”. In various embodiments, ensuring that the sequencing approach captures a broader spectrum of microbial genomes as well as produces longer contigs, one may increase the sequencing capture of accurate BGCs and significantly reduce the production of spurious BGCs and increase the capture of authentic BGCs. By capturing the authentic and primary DNA sequence as it is present in the patient sample, the system can increase the accuracy of subsequent analysis and capture strain-level evolution and horizontal gene transfer that occurs among microbes even as they depart from more outdated reference sequences.

The accuracy of BGC prediction can be dependent upon the quality and length of assembled contigs. To this point, the pipeline is optimized to perform de novo assembly of raw sequence data into contigs that reliably achieve lengths more than 20 kilobases and often up to megabase length. In various embodiments, this 20 kilobase cutoff is is utilized as a representation of a lower bound for the length of a BGC. Capturing and analyzing contigs shorter than 20 kilobases in various embodiments may result in dividing a BGC across separate contiguous stretches and thereby both failing to capture the BGC (false negative) and artifactually generating two putative BGCs that represent only subsets of an authentic BGC (false positive). By ensuring that the sequencing approach captures a broader spectrum of microbial genomes as well as produces longer contigs, one can increase the sequencing capture of accurate BGCs and significantly reduce the production of spurious BGCs.

As a de novo approach, the assignment of taxonomic labels and the use of marker genes or reference genomes may be deliberately avoided. By capturing the authentic and primary DNA sequence as it is present in the patient sample, one can increase the accuracy of subsequent analysis and capture strain-level evolution and horizontal transfer that occurs among microbes even as they depart from more outdated reference sequences.

De novo assembly and enhanced coverage of Applicant's optimized sequencing pipeline work in synergy to increase the identification of novel BGCs and concomitant molecular entities.

Once assembled, contigs may be systematically examined for evidence of synthetic potential, such as through the application of a Hidden Markov Model (HMM)-based algorithm. Regions of interest may be further annotated with gene and protein family labels, as examples.

Sequenced microbiome (FASTQ files) may be assembled using MegaHIT with a minimum k-mer size of 29 base pairs to produce contigs that are filtered to a minimum length of 20 kb. These assembled contigs can be annotated using Prodigal to predict Open Reading Frames that are subsequently annotated for membership in functional and metabolic pathways using the MetaPathways pipeline that makes use of homology search using ORFs against KEGG and RefSeq, in various embodiments.

Taxonomic Profiling

In optional cases, samples are taxonomically characterized using Kraken2 software package. Profiling of patient stool samples can process along both compositional and functional axes. For compositional analysis, taxonomic reports produced by Kraken2 can be analyzed using Bracken (Bayesian Reestimation of Abundance with Kraken). Bracken offers enhancement over raw taxonomic counts produced by Kraken in that it probabilistically re-distributes reads in the taxonomic tree, estimating species abundance in circumstances where Kraken may have opted for a lowest common ancestor at the genus level. This is a useful step in normalizing Kraken output for species taxonomic comparison. Having featurized each stool sample at species abundance tables, samples may be compared, such as using PCoA using Bray-Curtis distances to establish a Beta diversity metric between responders, non-responders, and interval sampling timepoints. As Bray-Curtis distances are non-phylogenetic, one can also calculate UniFrac distances and their resulting Beta diversity measurements. A goal of this compositional analysis can be to establish the broad compositional diversity between samples in responder and non-responder cohorts and to assess intra-subject microbiome variation overtime at different interval sampling points.

Extraction of Biosynthetic Gene Clusters

In particular embodiments, the platform is a tool for the systematic identification and selection of key groups of genes encoding proteins that produce uncharacterized metabolites. These groups of genes are BGCs that exist within the bacterial genomes that comprise the gut (including the stomach and/or intestines) microbiome. Many of the molecules that these bacteria produce act like drugs in that they interact with and elicit changes in the human immune system. The disclosure provides for identifying and characterizing these molecules and advancing them as novel therapeutics, in certain embodiments. In addition to compositional assessment, functional assessment of the metagenome with special focus on genes and Biosynthetic Gene Clusters (BGCs) may be performed.

Following gene assembly, the assembled contigs exceeding 20 kilobases in length undergo ORF, gene, and metabolic pathway annotations with identification of grouped adjacent genes known as Biosynthetic Gene Clusters (BGCs).

Explicit annotation of BGCs is performed using antiSMASH to produce gene-annotated complete BGC sequences with putative molecular product classes where applicable. As an optional step, one can use the deepBGC package to perform BGC identification and to compare its outputs against those of antiSMASH especially as enough samples are accumulated to perform specialized training of the deepBGC algorithm on study-specific data. These two tools perform similar functions but through very different approaches. Where antiSMASH is an HMM-based algorithm that can perform BGC identification with specificity, in at least some cases it tends to exclude novel BGCs that do not represent molecular classes on which it was trained. Thus, deepBGC's use of a Long Short-Term Memory Network may enable it to better identify novel BGCs and to benefit from extended algorithm training with relevant sample data.

Biostatistical Assessment

Samples may be compared in terms of their gene content, BGC content, taxonomic composition, and enrichment for specific metabolic pathways. To assess statistical significance between Responders and Non-Responders within subgroups (lung, skin, breast and kidney cancer), one can quantify the effect sizes in an interim analysis to limit confounding factors for a better design experiment. Various embodiments allow for avoiding confounding variables that have large effect sizes, such as diet and, age, and medium effect size, such as use of antibiotics. Antibiotics have a sustained effect on the microbiome, leading to altered community structure and lower alpha diversity.

It is often possible to see a clear separation between communities using Principal Coordinates Analysis (PCoA) space as a quick visualization technique for assessing large effects and small effects in a reduced-dimensionality space. However, statistical confirmation may be deemed useful. Effect size and statistical power can be challenging to calculate in microbiome data. One can utilize a PERMANOVA-based beta diversity comparison. The variation in beta diversity can be measured by pairwise distance based on either presence or abundance of species. PERMANOV A, a permutation-based extension of multivariate analysis of variance to a matrix of pairwise distances, partitions within-group and between-group distances to allow assessment of the effect of an exposure or intervention upon the sampled microbiome. PERMANOV A testing operates on distances, not species or OTU counts. Therefore, multiple different species distributions may serve to model the microbiome community structure.

To ensure adequate statistical power for each subgroup, the expected within-group variance and the effect to be expected from the predictor variables were quantified. Distance-based multivariate analysis of variance provides a non-parametric test of the null hypothesis of no differences in overall bacterial compositions among the Responder and Non-Responder groups. One can use UniFrac and Jaccard distances implemented in bioinformatic analysis packages. UniFrac method allows the incorporation of phylogenetic relationships among the taxa in the power calculation. Within-group variance depends upon the chosen distance metric and the metric may influence the observed effect.

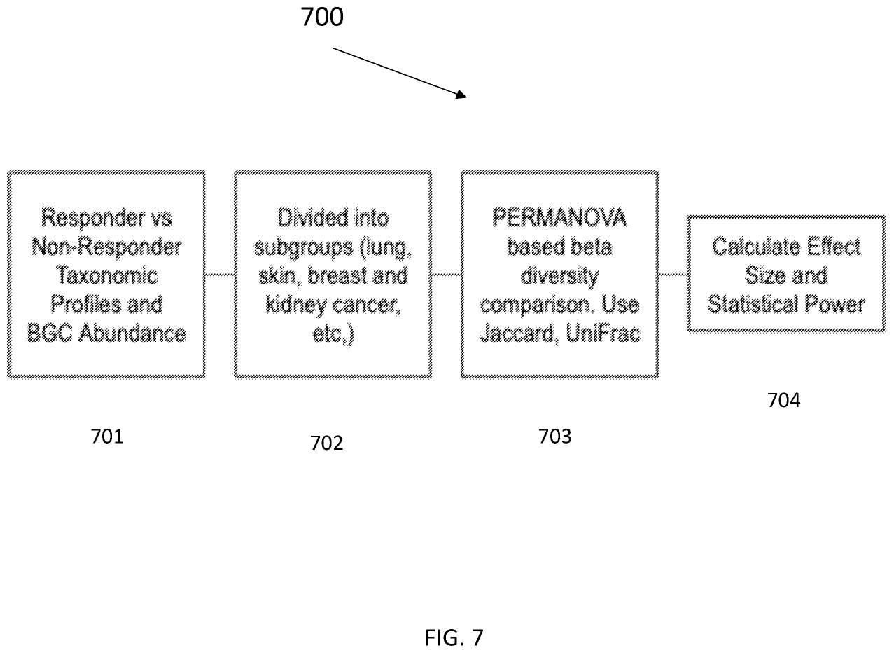

The proposed PERMANOV A method may be incorporated on BGCs as well. One can use the presence of the BGCs in samples in a similar fashion to an abundance of taxa for sample size and power calculations. This analysis of microbiome data can allow achievement of the goal of collecting rich metadata with an appropriate sample size while minimizing the technical variation during collection. FIG. 7 includes an example schematic workflow 700 illustrating the biostatistical assessment of a sample cohort using the PERMANOV A method, in accordance with various embodiments.

Topological Data Analysis

With BGCs, genes, taxa, and metabolic pathway participation having been featurized across samples, one can compare responder and non-responder groups including responder subgroups (e.g., complete response vs partial response) using two complementary approaches.

### Direct Graph-Based Network

Principal Component Analysis (PCA) may be used to explain the variance-covariance structure of a set of variables through linear combinations. It is often used as a dimensionality-reduction technique.

One such approach employs well-practiced multi-table methods such as concatenated PCA to identify BGCs and metabolic pathways driving response. As BGCs may be similar but non-identical, BGCs may be clustered according to Jaccard similarity allowing for a lower-dimensional and more dense representation of BGCs within which to perform PCA. This approach is analogous to matrix factorization techniques. In addition to this, one can also perform topological analyses of patient samples across various multi-omic data to include metabolomics (described below) as well as direct graph-based clustering of BGCs.

While this approach has been optimized for the disclosed platform, analogous methods have been employed in the literature. Specific similarity cut-offs often use-case specific therapeutic compounds constraints and are typically determined through iterative analysis of data to derive an algorithm that converges on an ideal BGC family cluster size and similarity. Importantly, the featurization approach is statistically defined and, thereby, is more flexible in its ability to accommodate BGCs and BGC families which are unique to the gut microbiome.

Direct graph-based BGC clustering involves the creation of a BGC similarity network wherein each node is an identified BGC and edges are drawn between nodes based upon several different similarity metrics that represent parity of gene identity, order, and duplication rate between BGCs. Iterative execution of this approach to calculate all pairs similarity produces a large data topology that captures connections between different BGCs—and families thereof—and their relationships with applicable metadata. Within the resulting similarity network, BGC “families” may be constructed as cliques of connected BGC nodes and ranked according to the enrichment of these cliques for samples from responder patient phenotypes. Initial identification of enriched cliques comprises calculating all enrichment percentages for cliques of three or more nodes and filtering for those nodes whose phenotypic enrichment is two or more standard deviations from the mean enrichment in either positive (responder-enriched) or negative (non-responder-enriched) direction.

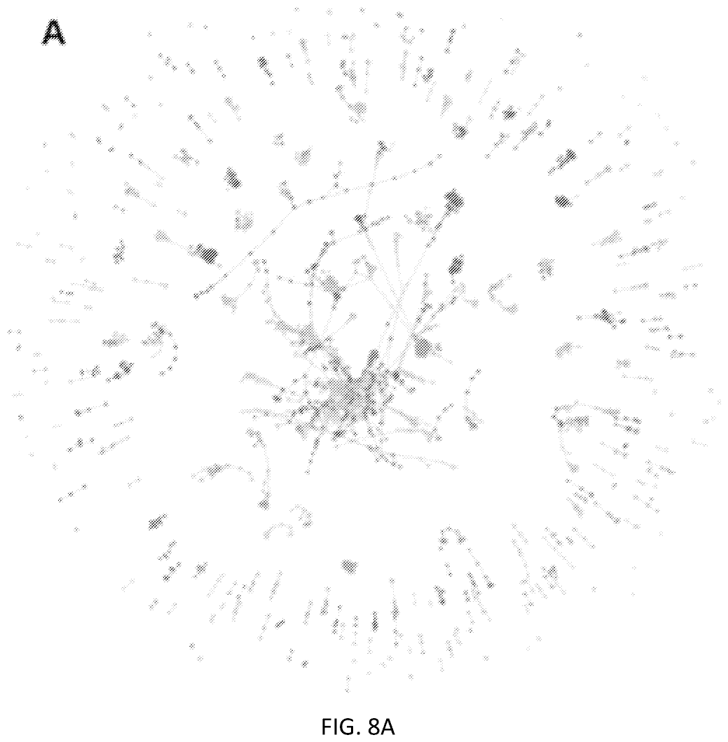

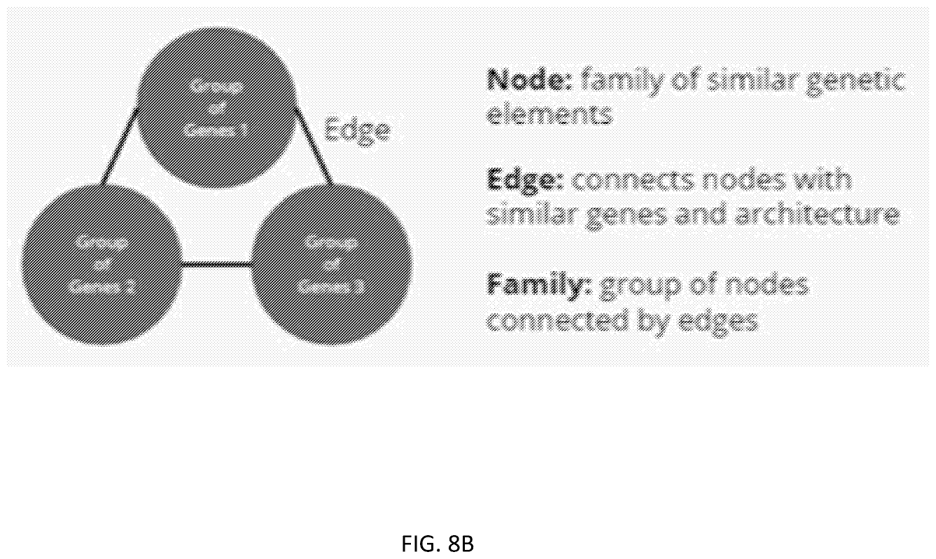

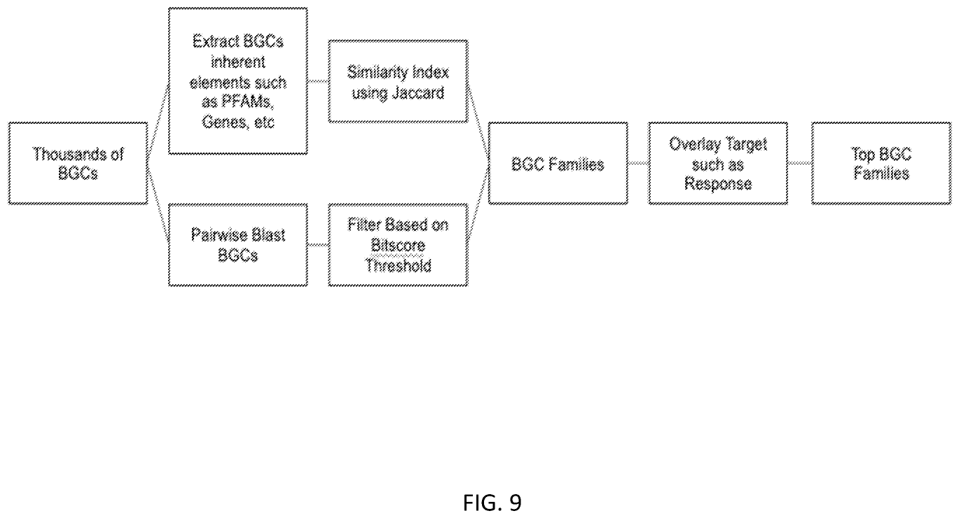

Once assembled, contigs are systematically examined for evidence of synthetic potential through the application of a Hidden Markov Model (HMM)-based algorithm that has been optimized for this purpose. Regions of interest are further annotated with gene and protein family (PFAM) labels. Candidate BGCs may then be clustered based on several different similarity metrics that represent parity of gene identity, order, and duplication rate between BGCs. Iterative execution of this approach to calculate all pairwise similarity produces a large data topology (referred to as a “similarity network”) that can capture connections between different BGCs—and families thereof—and their relationships with applicable metadata (see FIGS. 8A and 8B). FIG. 8, in particular, provides a visual representation of a BGC similarity network. In FIG. 8A, circles represent biosynthetic gene clusters (nodes) which are color coded based on metadata components. Lines (edges) connect these nodes that exceed a minimum threshold of similarity to form families. FIG. 8B, in particular, provides a schematic explanation of the relationship between nodes, edges and families. See FIG. 9 for an example schematic workflow illustrating a direct graph-based method, in accordance with various embodiments.

### Topological Data Analysis Network

Topological data analysis has shown value for exploratory data analysis and can be a powerful tool to identify separate subpopulations that may represent nonlinear influences on a target outcome such as therapeutic response. In short, the topological approach involved embedding high-dimensional data into a lower-dimensional space known as a simplicial complex the nodes and connections of which are based upon the distribution and partitioning of samples along with their various high-dimensional features, in various embodiments. This analytical approach produces a graph where nodes represent small subpopulations of samples and edges represent subsets of the original feature space connecting those samples. This graph may be used to “paint” nodes according to therapeutic response and time point-to-time point sampling to identify subsets of samples that may drive such outcomes for separate reasons. Identified subsets may then be compared against other control subsets to identify the most relevant dimensions driving such differences for each subpopulation.

Topological Data Analysis is a relatively new method of data exploration that has many unique strengths regarding hypothesis generation and exploration. This comparison includes the creation of a topological graph wherein each BGC occupies a node and inter-BGC similarity above minimum threshold results in the establishment of an edge. Cliques of related BGCs that are enriched in responder or non-responder phenotypes may be considered targets for further investigation.

As mentioned previously, related molecular structures and related BGCs may both be determined topologically as cliques within their respective graphs. In addition to this determination, these cliques are assessed for phenotypic enrichment according to whether participant nodes were identified from responder or non-responder samples. Initial identification of enriched cliques may comprise calculating all enrichment percentages for cliques of three or more nodes and filtering for those nodes whose phenotypic enrichment is two or more standard deviations from the mean enrichment in either positive (responder-enriched) or negative (non-responder-enriched) direction.

Candidate BGCs may then be clustered based on several different metrics and lens functions. Iterative execution of this approach to calculate all pairs similarity produces a large data topology that captures connections between different BGCs and their relationships with applicable metadata. The similarity network acts, therefore, as an unsupervised clustering approach for BGCs and can identify BGC families without prior knowledge of the expected BGC sequence or architecture. Gene clusters may then be grouped by homology, and each group of related BGCs is scored based upon the clinical response to immunotherapy exhibited by the subject from whose sample the gene cluster was extracted.

In case of the possibility of collecting and analyzing plasma and stool for metabolomic signatures, additional analysis involves tandem mass spectrometry resulting in mass spectra for each sample reflecting the abundance and structure of identified metabolites. Work within these datasets seeks to determine which mass spectra is most attributable to the patient response by comparing responder and non-responder groups using multi-table methods mentioned above. The connection between identified metabolites and BGCs to which one can perform covariation analyses of BGC and metabolites may feature spaces to identify which BGCs may produce which metabolites and, especially, which BGCs can produce metabolites that are detectable in-patient sera. These data may also be used as additional dimensions in the feature space of topological data analysis which identifies subpopulation among both responder and non-responder groups that have both BGC and metabolite features in common.

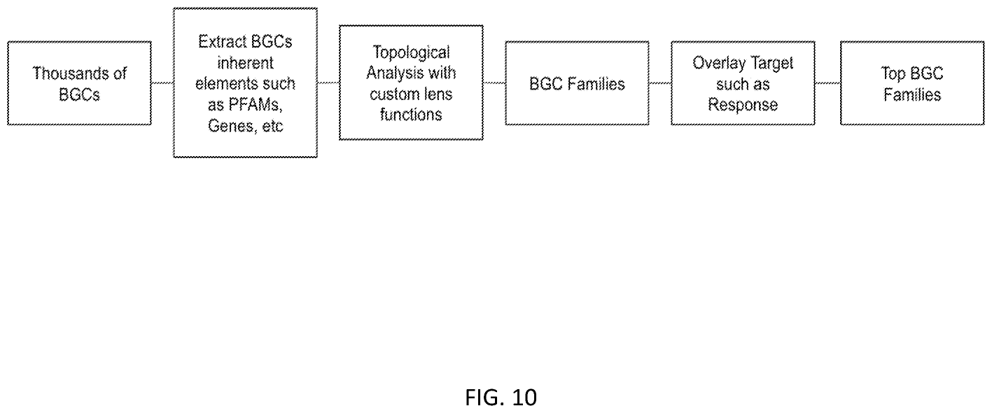

See FIG. 10 for an example schematic workflow illustrating topological data analysis process, in accordance with various embodiments.

ML Model

Following clustering optimization, BGCs families may further be used as predictors for an artificial intelligence model (e.g., a machine-learning model) to assess clinical outcomes in checkpoint therapy patients. The ML model can integrate features and clusters upstream and ranks BGCs for downstream lab experiments. The ML model is also capable of predicting patient outcomes using these BGCs and utilizing them as potential biomarkers.

Many artificial intelligence approaches treat deep learning models as a “black box”. In these cases, users often rely on functional performance of the model with its application limited to the specific functional use case in which it was trained. The value of deep learning—especially in the life sciences—runs much deeper when models can be explored not just as discriminators and predictors but also as hypothesis generation tools. Since detailed a priori causal hypotheses are often lacking in biological use cases, being able to interrogate a performant trained deep learning algorithm can be a very useful technique to bootstrap subsequent analysis.

The machine learning models constitute one piece of a multi-layered approach that synergistically combines advanced deep learning techniques, extensive domain expertise (held within the core team and an extended advisory network), and publicly available data to improve the prediction and prioritization of influential BGCs. The machine learning algorithm not only achieves high accuracy when interpreting BGCs and predicting clinical outcome, but learned feature importance can serve as a valuable starting point to implicate specific, clinically important BGCs which go on to further review by human expertise.

A unique deep learning algorithm has been developed to predict outcomes in ICI therapy using metagenomic data. The genetic components identified in stool samples may be organized based on their sub elements and used as predictors for this deep learning algorithm. The classification algorithm employs convolutional neural networks combined with methods used in natural language processing. This classification can be in the form of broad clinical outcomes like response to ICI therapy. However, given the wealth of associated metadata, classification can occur at a much more granular level (for example, the degree of tumor infiltration by lymphocytes, or circulating interleukin levels), allowing the platform to begin to reason around potential mechanistic insights.

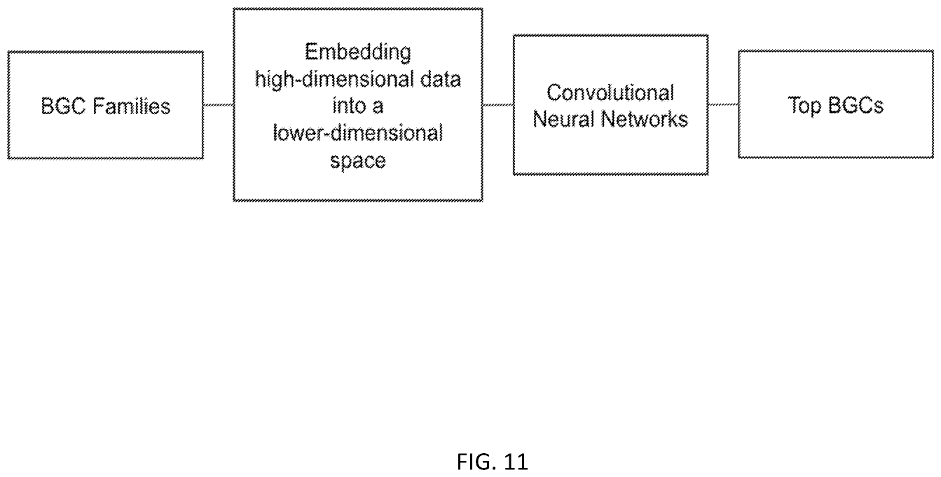

See FIG. 11 for an example schematic workflow illustrating a machine learning workflow, in accordance with various embodiments. This process can be used, for example, to integrate features and clusters upstream and ranks BGCs for downstream lab experiments.

To assess BGC importance, BGC features are successively permuted and excluded from the trained model. The model is then reiteratively tested with certain features “dropped out” and those with the greatest functional impact on model performance are ranked higher. Gene clusters implicated in this way are then aligned to known bacterial genomes, many of which have been experimentally shown to have immunomodulatory effects in mammalian systems. This readily facilitates preliminary validation of candidate BGCs and can provide context to inform experimental design wherein molecular products of these BGCs will be generated and tested.

Preliminary work suggests that this system performs considerably better than other published approaches that use common taxonomic classification and traditional machine learning models to classify microbiome data. In fact, the performance of the Applicant model approaches that of many diagnostic tests in clinical use. These results provide a list of prioritized genetic signatures that may lead to the development of biomarkers to be used to predict clinical outcomes for ICIs.

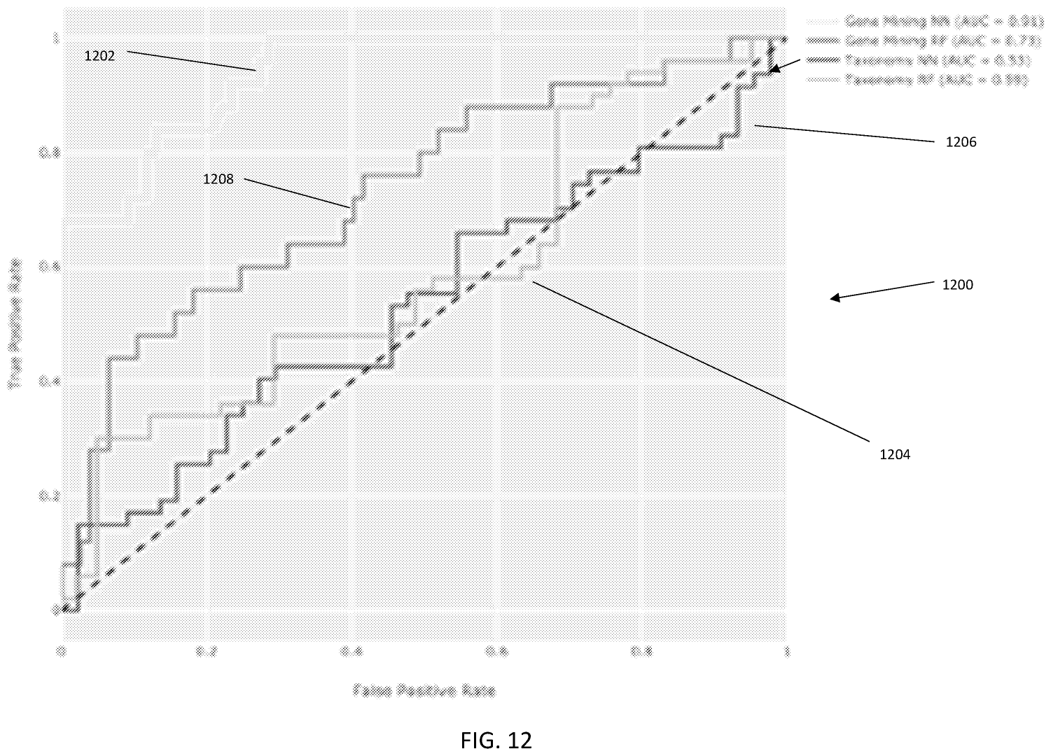

FIG. 12 provides a plot 1200 of the performance of an applicant model for predicting outcomes in ICI therapy, in accordance with various embodiments. The model outperforms traditional machine learning methods trained on data featurized by taxonomic classification and gene mining. The deep learning model can also be significantly more accurate when trained on whole genome shotgun inputs compared to marker genes (16S data). FIG. 12 provides a receiver operating characteristic (ROC) curve illustrating the superior performance when trained on BGC-derived features (1202) compared to model trained on marker-gene-derived features (1204), traditional machine learning methods trained on BGC-derived features (1206), and traditional machine learning methods trained on marker-gene-derived features (1208).

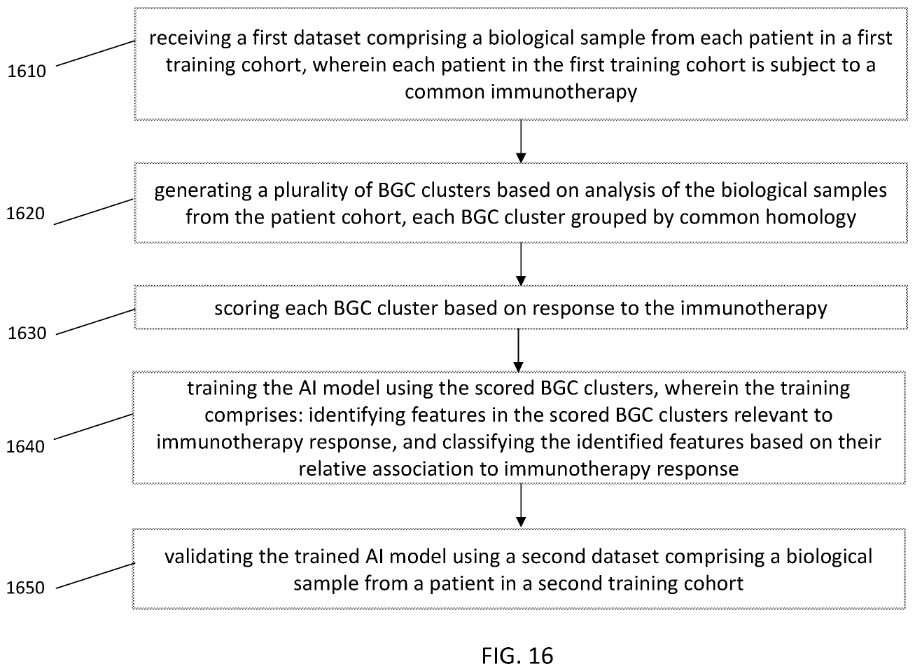

In accordance with various embodiments, and as illustrated for example in FIG. 16, a method is provided for training an artificial intelligence (AI) model for assessing or predicting clinical outcomes in immunotherapy patients using identified microbial biosynthetic genetic cluster (BGCs). As provided in step 1610, the method can comprise receiving a first dataset comprising a biological sample from each patient in a first training cohort, wherein each patient in the first training cohort is subject to a common immunotherapy. As provided in step 1620, the method can comprise generating a plurality of BGC clusters based on analysis of the biological samples from the patient cohort, each BGC cluster grouped by common homology. As provided in step 1630, the method can comprise scoring each BGC cluster based on response to the immunotherapy. As provided in step 1640, the method can comprise training the AI model using the scored BGC clusters, wherein the training comprises identifying features in the scored BGC clusters relevant to immunotherapy response, and classifying the identified features based on their relative association to immunotherapy response). As provided in step 1650, the method can comprise validating the trained AI model using a second dataset comprising a biological sample from a patient in a second training cohort.

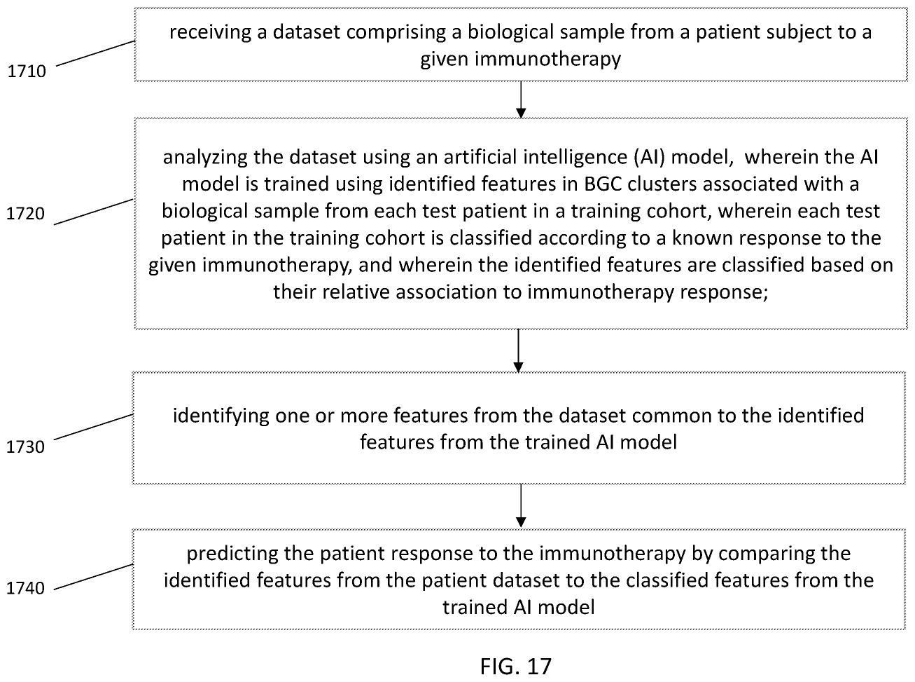

In accordance with various embodiments, and as illustrated for example in FIG. 17, a method is provided for assessing or predicting clinical outcomes in immunotherapy patients using identified microbial biosynthetic genetic cluster (BGCs). As provided in step 1710, the method can comprise receiving a dataset comprising a biological sample from a patient subject to a given immunotherapy. As provided in step 1720, the method can comprise analyzing the dataset using an artificial intelligence (AI) model, wherein the AI model is trained using identified features in BGC clusters associated with a biological sample from each test patient in a training cohort, wherein each test patient in the training cohort is classified according to a known response to the given immunotherapy, and wherein the identified features are classified based on their relative association to immunotherapy response. As provided in step 1730, the method can comprise identifying one or more features from the dataset common to the identified features from the trained AI model. As provided in step 1740, the method can comprise predicting the patient response to the immunotherapy by comparing the identified features from the patient dataset to the classified features from the trained AI model.

In accordance with various embodiments, the AI model may be a machine learning model. In accordance with various embodiments, the immunotherapy is a checkpoint therapy. In accordance with various embodiments, the biological sample comprises a stool sample or a plasma sample. In accordance with various embodiments, the classifying comprises ranking the identified features based on their relative association to immunotherapy response. In accordance with various embodiments, the classifying comprises using a neural network, natural language processing, or a combination thereof.

In accordance with various embodiments, provided is a non-transitory computer-readable medium storing computer instructions that, when executed by a computer, cause the computer to perform a method for training an artificial intelligence (AI) model for assessing or predicting clinical outcomes in immunotherapy patients using identified microbial biosynthetic genetic cluster (BGCs), the method comprising: receiving a first dataset comprising a biological sample from each patient in a first training cohort, wherein each patient in the first training cohort is subject to a common immunotherapy; generating a plurality of BGC clusters based on analysis of the biological samples from the patient cohort, each BGC cluster grouped by common homology; scoring each BGC cluster based on response to the immunotherapy; training the AI model using the scored BGC clusters, wherein the training comprises: identifying features in the scored BGC clusters relevant to immunotherapy response, and classifying the identified features based on their relative association to immunotherapy response); and validating the trained AI model using a second dataset comprising a biological sample from a patient in a second training cohort.

In accordance with various embodiments, provided is a non-transitory computer-readable medium storing computer instructions that, when executed by a computer, cause the computer to perform a method for assessing or predicting clinical outcomes in immunotherapy patients using identified microbial biosynthetic genetic cluster (BGCs), the method comprising: receiving a dataset comprising a biological sample from a patient subject to a given immunotherapy; analyzing the dataset using an artificial intelligence (AI) model, wherein the AI model is trained using identified features in BGC clusters associated with a biological sample from each test patient in a training cohort, wherein each test patient in the training cohort is classified according to a known response to the given immunotherapy, and wherein the identified features are classified based on their relative association to immunotherapy response; identifying one or more features from the dataset common to the identified features from the trained AI model, and predicting the patient response to the immunotherapy by comparing the identified features from the patient dataset to the classified features from the trained AI model.

Computer Implemented System

In various embodiments, the methods for generating cell populations and non-cell populations from a multi genomic feature sequence dataset for joint cell calling can be implemented via computer software or hardware. That is, the methods disclosed herein can be implemented on a computing device that can include at least or more of processing and analytical engines. In various embodiments, the computing device can be communicatively connected to a data storage unit and a display device via a direct connection or through an internet connection.

It should be appreciated that the various engines that can be used to help execute the various embodiments herein, can be combined or collapsed into a single engine, component or module, depending on the requirements of the particular application or system architecture. Moreover, in various embodiments, the processing and analytical engines can comprise additional engines or components as needed by the particular application or system architecture.

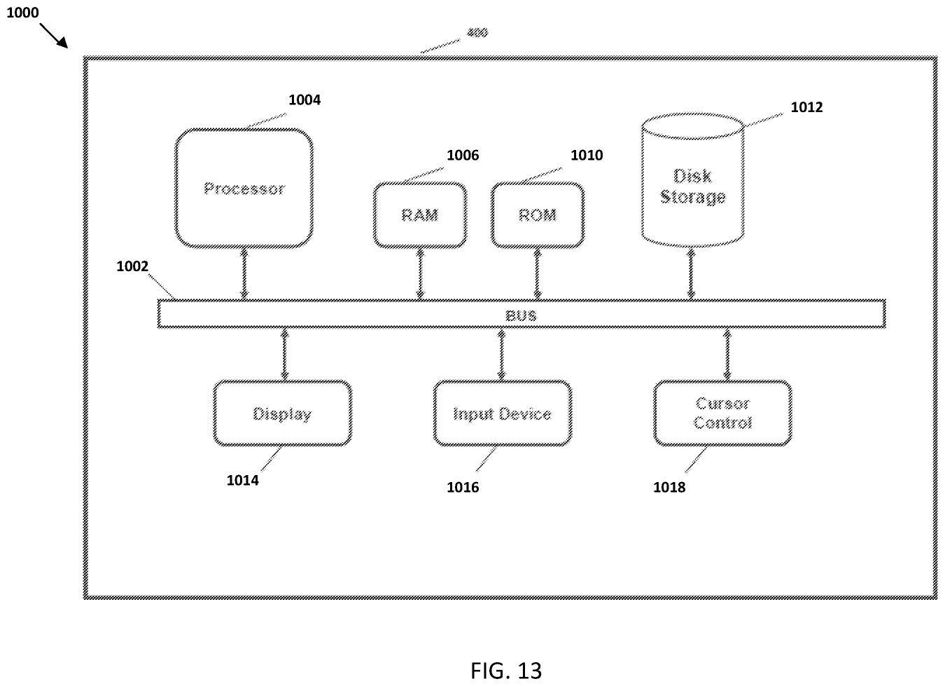

FIG. 13 is a block diagram illustrating an example of a computer system 1000 upon which embodiments of the present teachings may be implemented. In various embodiments of the present teachings, computer system 1000 can include a bus 1002 or other communication mechanism for communicating information and a processor 1004 coupled with bus 1002 for processing information. In various embodiments, computer system 1000 can also include a memory, which can be a random-access memory (RAM) 1006 or other dynamic storage device, coupled to bus 1002 for determining instructions to be executed by processor 1004. Memory can also be used for storing temporary variables or other intermediate information during execution of instructions to be executed by processor 1004. In various embodiments, computer system 1000 can further include a read only memory (ROM) 1010 or other static storage device coupled to bus 1002 for storing static information and instructions for processor 1004. A storage device 1012, such as a magnetic disk or optical disk, can be provided and coupled to bus 1002 for storing information and instructions.

In various embodiments, computer system 1000 can be coupled via bus 1002 to a display 1014, such as a cathode ray tube (CRT) or liquid crystal display (LCD), for displaying information to a computer user. An input device 1016, including alphanumeric and other keys, can be coupled to bus 1002 for communication of information and command selections to processor 1004. Another type of user input device is a cursor control 1018, such as a mouse, a trackball or cursor direction keys for communicating direction information and command selections to processor 1004 and for controlling cursor movement on display 1014. This input device 1016 typically has two degrees of freedom in two axes, a first axis (i.e., x) and a second axis (i.e., y), that allows the device to specify positions in a plane. However, it should be understood that input devices 1016 allowing for 3-dimensional (x, y and z) cursor movement are also contemplated herein.

Consistent with certain implementations of the present teachings, results can be provided by computer system 1000 in response to processor 1004 executing one or more sequences of one or more instructions contained in memory 1006. Such instructions can be read into memory 1006 from another computer-readable medium or computer-readable storage medium, such as storage device 1012. Execution of the sequences of instructions contained in memory 1006 can cause processor 1004 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, dynamic memory, such as memory 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 1000 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1000, whereby processor 1004 would execute the analyses and determinations provided by these engines, subject to instructions provided by any one of, or a combination of, memory components 1006/1010/1012 and user input provided via input device 1016.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

### EMBODIMENTS OF METHODS OF USE

The present disclosure provides systems and compositions that are suitable for a variety of methods of use of the system or parts thereof. The disclosure provides for methods of identifying particular microbial BGCs, methods for ranking microbial BGCs, method of identifying microbial BGCs related to a response to a therapy in an individual, and so forth. The different methods may or may not have more than one overlapping step. The methods may or may not have similar purposes. Some methods may identify a therapy suitable for an individual, whereas other methods produce systems for such identification, whereas other methods are utilized for drug development based on known association with an effective response.

In one embodiment, the methods may comprise steps such as sequencing microbial DNA; collating sequencing information; identifying BGCs; sequencing BGCs assigning BGCs based on origination of the source; ranking BGCs comparing BGCs based on sequence; denoting BGCs as being from responders or from non-responders; correlating BGCs to a standard, and so forth.

In one embodiment, there is a method of identifying microbial BGCs (including at least bacterial DNA) related to a response to a therapy in an individual, comprising (a) sequencing microbial DNA from samples from a plurality of individuals having received a therapy, wherein a first group of individuals are responders to the therapy and a second group of individuals are non-responders to the therapy; (b) collating sequencing information from each of the plurality of individuals to produce contiguous sequencing reads (contigs; such as no less than about 20K base pairs in length); (c) identifying BGCs from the respective contigs; (d) assigning BGCs as originating from the first group of individuals or the second group of individuals; and (e) ranking the BGCs according to statistical association to a responder phenotype. As used herein, the term “responder phenotype” in any method encompassed herein includes a clinical response in which an individual having received a therapy had complete response, partial response, or stable disease for at least 12 months, as conditions well known in the art. In specific embodiments, the sample is from the gut, from blood, from stool, or a mixture thereof. In certain embodiments, DNA of the individual(s) in the plurality is not sequenced or not intended to be sequenced. The therapy may be of any kind, but in specific cases it is cancer therapy, including immunotherapy, such as immune checkpoint immunotherapy. In specific embodiments, the method further comprises the step of comparing one or more BGC sequences from an individual having an unknown response to the therapy to the ranked BGCs, thereby determining an indication of a response to the therapy for the individual. When the individual is considered to have one or more BGC sequences associated with a responder phenotype, the individual is given the therapy, and when the individual is considered not to have one or more BGC sequences associated with a responder phenotype, the individual is not given the therapy but may be administered a therapeutically effective amount of one or more therapies that are not immune checkpoint immunotherapies.

In various embodiments, there is a method of identifying microbial biosynthetic genetic cluster (BGCs) related to a response to a therapy in an individual, the method comprising: (a) sequencing microbial DNA from samples from a plurality of individuals having received a therapy, wherein a first group of individuals are responders to the therapy and a second group of individuals are non-responders to the therapy; (b) collating sequencing information from each of the plurality of individuals to produce contiguous sequencing reads (contigs); (c) identifying one or more BGCs from the respective contigs; (d) assigning BGCs as originating from the first group of individuals or the second group of individuals; and (e) ranking the BGCs according to statistical association to a responder phenotype. In various embodiments, the microbial DNA may be bacterial DNA. The sample may be from the gut, from blood, from stool, or a mixture thereof. In various embodiments, DNA of one or more of the individuals in the plurality is not sequenced or not intended to be sequenced, which may be by whole genome shotgun sequencing. In various embodiments, at least the majority of the contigs are no less than about 20K base pairs in length. The therapy may be cancer therapy, such as immunotherapy, including at least immune checkpoint immunotherapy (such as anti-programmed cell death protein 1 (PD1) therapy, anti-Programmed death-ligand 1 (PD-L1) therapy, anti-cytotoxic T-lymphocyte-associated antigen 4 (CTLA-4) therapy, or a combination thereof). The responders may be complete responders or partial responders. The method may further comprise the step of comparing one or more BGC sequences from a gut microbe from an individual having an unknown response to the therapy to the ranked BGCs, thereby determining an indication of a response to the therapy for the individual. When the individual may be considered to have one or more BGC sequences associated with a responder phenotype, the individual may be given the therapy. When the individual is considered not to have one or more BGC sequences associated with a responder phenotype, the individual may not be given the therapy and in some cases may instead be administered a therapeutically effective amount of one or more therapies that are not immune checkpoint immunotherapies.

In various embodiments, there are methods of determining a suitable treatment for an individual. In various embodiments, there are methods of determining a treatment regimen for an individual in need of a therapy, comprising the step of comparing the sequence of one or more BGCs from gut microbes of the individual to a system that ranks BGCs according to response or non-response to the therapy. In such cases, when the one or more BGCs from microbes of the individual correlate to BGCs from the system associated with a response to the therapy, the individual is administered an effective amount of the therapy, and when the one or more BGCs from microbes of the individual correlate to BGCs from the system associated with non-response to the therapy, the individual is not administered the therapy. Such a system may or may not be produced by certain methods. In various embodiments, the system is produced by analyzing BGCs from a plurality of individuals having received the therapy and that were responders or non-responders, followed by ranking of the BGCs according to statistical association to a responder phenotype. Production of the system may comprise: (a) sequencing microbial gut DNA from the plurality of individuals to produce sequencing reads; (b) aligning sequencing reads into contigs of no less than 20K base pairs; (c) identifying the BGCs based on their sequence and grouping BGCs of similar sequence; (d) denoting BGCs as being from responders or from non-responders; and (e) ranking statistically the BGCs from responders.

Methods of determining a treatment regimen using any system encompassed herein are contemplated. The treatment regimen may concern immunotherapy, such as at least immune checkpoint immunotherapy, including anti-programmed cell death protein 1 (PD1) therapy, anti-Programmed death-ligand 1 (PD-L1) therapy, anti-cytotoxic T-lymphocyte-associated antigen 4 (CTLA-4) therapy, or a combination thereof. In various embodiments, there is a method of determining a treatment regimen for an individual in need of a therapy, comprising the step of comparing the sequence of one or more BGCs from gut microbes of the individual to a system that ranks BGCs according to response or non-response to the therapy. In various embodiments, when the one or more BGCs from microbes of the individual correlate to BGCs from the system associated with a response to the therapy, the individual is administered an effective amount of the therapy. In various embodiments, when the one or more BGCs from microbes of the individual correlate to BGCs from the system associated with non-response to the therapy, the individual is not administered the therapy. The system may be of any kind but may be produced by analyzing BGCs from a plurality of individuals having received the therapy and that were responders or non-responders, followed by ranking of the BGCs according to statistical association to a responder phenotype. In specific cases, production of the system may comprise: (a) sequencing microbial gut DNA from the plurality of individuals to produce sequencing reads; (b) aligning sequencing reads into contigs of no less than 20K base pairs; (c) identifying the BGCs based on their sequence and grouping BGCs of similar sequence; (d) denoting BGCs as being from responders or from non-responders; and (e) ranking statistically the BGCs from responders.

Methods of treatment are encompassed herein. In various embodiments, an individual in need of treatment is administered a therapeutically effective amount of the therapy for which they are being tested for efficacy. In other cases when their test for efficacy for the therapy is not determined to be efficacious, they are administered a different therapy. In various embodiments, there is a method of treating an individual in need thereof, comprising the step of administering a therapeutically effective amount of a therapy to the individual that has one or more BGCs from gut microbes that are indicative of response to the therapy. The method may further comprise comparing the sequence of one or more BGCs from gut microbes from the individual to the sequence of one or more BGCs from gut microbes from a plurality of individuals each having a known response or known non-response to the therapy. Treatment may be administered to the individual when the sequence of one or more BGCs from gut microbes from the individual correlates to sequence of one or more BGCs from gut microbes from individuals having a response to the therapy. In various embodiments, the therapy comprises one or more immune checkpoint immunotherapies.

In various embodiments, there is a method of treating an individual in need thereof, comprising the step of administering a therapeutically effective amount of a therapy (such as one or more immune checkpoint immunotherapies) to an individual that has one or more BGCs from gut microbes that are indicative of response to the therapy. The method may further comprising comparing the sequence of one or more BGCs from gut microbes from the individual to known sequences in a system or database, including sequences of one or more BGCs from gut microbes from a plurality of individuals each having a known response or known non-response to that therapy. In various embodiments, treatment is administered to the individual when the sequence of one or more BGCs from gut microbes from the individual correlates in a system or database to sequence of one or more BGCs from gut microbes from individuals having a response to the therapy. In so various me embodiments, there is a method of developing a therapy of any kind, comprising the steps of identifying one or more metabolites produced from one or more BGCs from gut microbes from one or more individuals, wherein the BGCs are associated with a responder phenotype to a therapy; and testing the one or more metabolites for efficacy as the therapy. The testing may be of any kind and may be in vitro, ex vivo, or in vivo. The testing may be for activity as an immune checkpoint inhibitor. The testing may be for activity against PD1, PD-L1, and/or CTLA-4. In some cases, the one or more metabolites are considered to be lead compounds for drug development and may be further modified, such as alteration of one or more R groups on the one or more metabolites.

In various embodiments, there is a method of developing a therapy, comprising the steps of: identifying one or more metabolites produced from one or more BGCs from gut microbes from one or more individuals, wherein the BGCs are associated with a responder phenotype to a therapy; and testing (such as in vitro, ex vivo, or in vivo) the one or more metabolites for efficacy as the therapy. The activity being tested for the one or more metabolites may be of any kind, but in specific cases the activity being tested is for activity for inhibition of any kind against any immune checkpoint inhibitor, not limited to but for example, PD1, PD-L1, and/or CTLA-4. Upon identification of the one or more metabolites having the desired activity, they may be further modified in any manner.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Prediction of Response Outcome Using the Platform

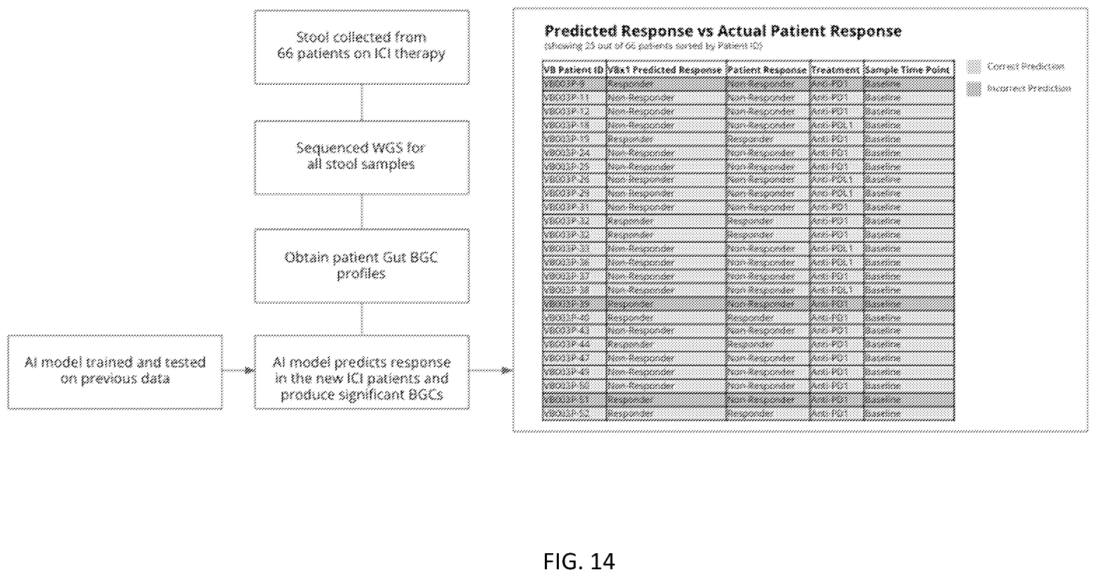

The novel bioinformatics pipeline and AI algorithm of FIG. 2 was applied to a new set of patients from a cancer center in Florida, USA. The patient cohort consisted of 66 patients undergoing ICI therapy and that lacked a known response to the ICI therapy. Their samples were applied to the system of the disclosure, and the system produced an accurate response status for 54 patients as shown in FIG. 14.

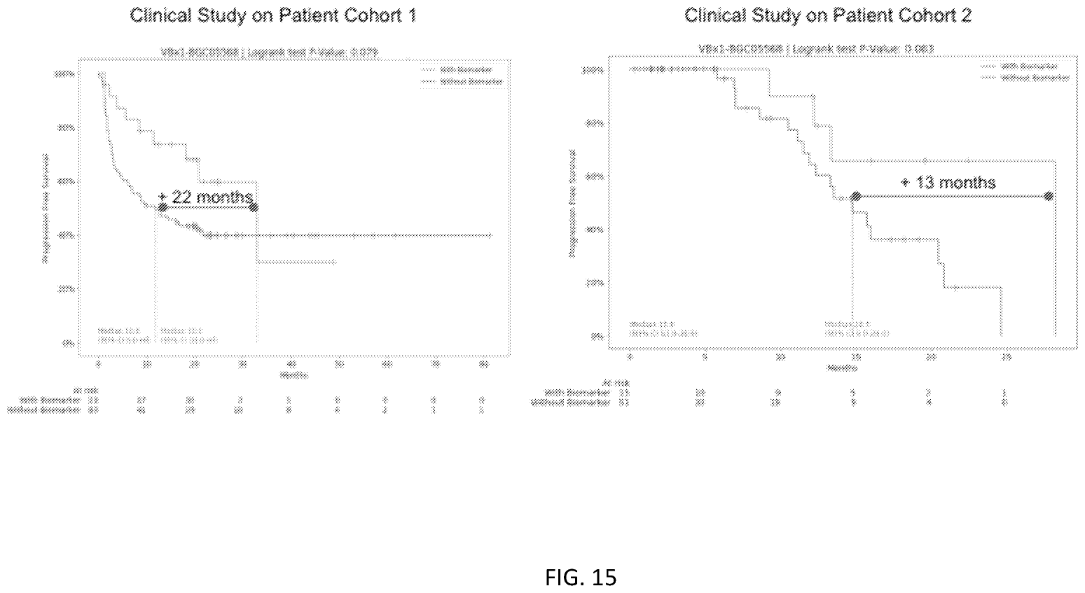

The system has produced remarkable BGCs that are shown to stratify patients response in several cohorts. As an example, the presence of particular BGC in patient's gut in FIG. 15 is shown to increase life by 13 and 22 months in Patient Cohort 1 and Patient Cohort 2, respectively. It is also shown to correlate strongly with Response in two separate studies (Patient Cohort 3 and Patient Cohort 4). This indicates that this particular BGC identified by the system can be used as biomarker or a starting therapeutic lead for ICI therapy.

### REFERENCES

The following references, to the extent that they provide exemplary procedural or other details supplementary to those set forth herein, are specifically incorporated herein by reference.
Darvin et al. 2018 Darvin P, Toor S M, Sasidharan Nair V, Elkord E. Immune checkpoint inhibitors: recent progress and potential biomarkers. Exp Mol Med. 2018 Dec. 13; 50(12):1-11. doi: 10.1038/s12276-018-0191-1. PMID: 30546008; PMCID: PMC6292890.
Gopalakrishnan et al. 2017 Gopalakrishnan V, Spencer C N, Nezi L, Reuben A, Andrews M C, Karpinets T V, Prieto P A, Vicente D, Hoffman K, Wei S C, Cogdill A P, Zhao L, Hudgens C W, Hutchinson D S, Manzo T, Petaccia de Macedo M, Cotechini T, Kumar T, Chen W S, Reddy S M, Szczepaniak Sloane R, Galloway-Pena J, Jiang H, Chen P L, Shpall E J, Rezvani K, Alousi A M, Chemaly R F, Shelburne S, Vence L M, Okhuysen P C, Jensen V B, Swennes A G, McAllister F, Marcelo Riquelme Sanchez E, Zhang Y, Le Chatelier E, Zitvogel L, Pons N, Austin-Breneman J L, Haydu L E, Burton E M, Gardner J M, Sirmans E, Hu J, Lazar A J, Tsujikawa T, Diab A, Tawbi H, Glitza I C, Hwu W J, Patel S P, Woodman S E, Amaria R N, Davies M A, Gershenwald J E, Hwu P, Lee J E, Zhang J, Coussens L M, Cooper Z A, Futreal P A, Daniel C R, Ajami N J, Petrosino J F, Tetzlaff M T, Sharma P, Allison J P, Jenq R R, Wargo J A. Gut microbiome modulates response to anti-PD-1 immunotherapy in melanoma patients. Science. 2018 Jan. 5; 359(6371):97-103. doi: 10.1126/science.aan4236. Epub 2017 Nov. 2. PMID: 29097493; PMCID: PMC5827966.
Lu et al. Lu E, Shatzel J, Shin F, Prasad V. What constitutes an “unmet medical need” in oncology? An empirical evaluation of author usage in the biomedical literature. Semin Oncol. 2017 February; 44(1):8-12. doi: 10.1053/j.seminoncol.2017.02.009. Epub 2017 Feb. 9. PMID: 28395768.
Matson et al. 2018 Matson V, Chervin C S, Gajewski T F. Cancer and the Microbiome-Influence of the Commensal Microbiota on Cancer, Immune Responses, and Immunotherapy. Gastroenterology. 2021; 160(2):600-613. doi:10.1053/j.gastro.2020.11.041
Routy et al. 2017 Routy B, Le Chatelier E, Derosa L, Duong CPM, Alou M T, Daillère R, Fluckiger A, Messaoudene M, Rauber C, Roberti M P, Fidelle M, Flament C, Poirier-Colame V, Opolon P, Klein C, Iribarren K, Mondragón L, Jacquelot N, Qu B, Ferrere G, Clemenson C, Mezquita L, Masip J R, Naltet C, Brosseau S, Kaderbhai C, Richard C, Rizvi H, Levenez F, Galleron N, Quinquis B, Pons N, Ryffel B, Minard-Colin V, Gonin P, Soria J C, Deutsch E, Loriot Y, Ghiringhelli F, Zalcman G, Goldwasser F, Escudier B, Hellmann M D, Eggermont A, Raoult D, Albiges L, Kroemer G, Zitvogel L. Gut microbiome influences efficacy of PD-1-based immunotherapy against epithelial tumors. Science. 2018 Jan. 5; 359(6371):91-97. doi: 10.1126/science.aan3706. Epub 2017 Nov. 2. PMID: 29097494.
Vetizou et al. 2015 Vétizou M, Pitt J M, Daillere R, Lepage P, Waldschmitt N, Flament C, Rusakiewicz S, Routy B, Roberti M P, Duong C P, Poirier-Colame V, Roux A, Becharef S, Formenti S, Golden E, Cording S, Eberl G, Schlitzer A, Ginhoux F, Mani S, Yamazaki T, Jacquelot N, Enot D P, Bérard M, Nigou J, Opolon P, Eggermont A, Woerther P L, Chachaty E, Chaput N, Robert C, Mateus C, Kroemer G, Raoult D, Boneca I G, Carbonnel F, Chamaillard M, Zitvogel L. Anticancer immunotherapy by CTLA-4 blockade relies on the gut microbiota. Science. 2015 Nov. 27; 350(6264):1079-84. doi: 10.1126/science.aad1329. Epub 2015 Nov. 5. PMID: 26541610; PMCID: PMC4721659.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### RECITATION OF EMBODIMENTS

Embodiment 1: A method of identifying microbial biosynthetic genetic cluster (BGCs) related to a response to a therapy in an individual, the method comprising: (a) sequencing microbial DNA from samples from a plurality of individuals having received a therapy, wherein a first group of individuals are responders to the therapy and a second group of individuals are non-responders to the therapy; (b) collating sequencing information from each of the plurality of individuals to produce contiguous sequencing reads (contigs); (c) identifying one or more BGCs from the respective contigs; (d) assigning BGCs as originating from the first group of individuals or the second group of individuals; and (e) ranking the BGCs according to statistical association to a responder phenotype.

Embodiment 2: The method of Embodiment 1, wherein the microbial DNA is bacterial DNA.

Embodiment 3: The method of Embodiments 1 or 2, wherein the sample is from the gut, from blood, or a mixture thereof.

Embodiment 4: The method of any one of the preceding Embodiments, wherein the sample is from the stool.

Embodiment 5: The method of any one of the preceding Embodiments, wherein DNA of the individuals in the plurality is not sequenced or not intended to be sequenced.

Embodiment 6: The method of any one of the preceding Embodiments, wherein the sequencing is by whole genome shotgun sequencing.

Embodiment 7: The method of any one of the preceding Embodiments, wherein at least the majority of the contigs are no less than about 20K base pairs in length.

Embodiment 8: The method of Embodiments 1 or 2, wherein the therapy is cancer therapy.

Embodiment 9: The method of Embodiment 3, wherein the cancer therapy is immunotherapy.

Embodiment 10: The method of Embodiment 4, wherein the immunotherapy is immune checkpoint immunotherapy.

Embodiment 11: The method of Embodiment 5, wherein the immune checkpoint immunotherapy is anti-programmed cell death protein 1 (PD1) therapy, anti-Programmed death-ligand 1 (PD-L1) therapy, anti-cytotoxic T-lymphocyte-associated antigen 4 (CTLA-4) therapy, or a combination thereof.

Embodiment 12: The method of Embodiment 1, wherein the responders are complete responders or partial responders.

Embodiment 13: The method of any one of the preceding Embodiments, further comprising the step of comparing one or more BGC sequences from a gut microbe from an individual having an unknown response to the therapy to the ranked BGCs, thereby determining an indication of a response to the therapy for the individual.

Embodiment 14: The method of Embodiment 13, wherein when the individual is considered to have one or more BGC sequences associated with a responder phenotype, the individual is given the therapy.

Embodiment 15: The method of Embodiment 13, wherein when the individual is considered not to have one or more BGC sequences associated with a responder phenotype, the individual is not given the therapy.

Embodiment 16: The method of Embodiment 15, wherein the individual is administered a therapeutically effective amount of one or more therapies that are not immune checkpoint immunotherapies.

Embodiment 17: A method of determining a treatment regimen for an individual in need of a therapy, comprising the step of comparing the sequence of one or more BGCs from gut microbes of the individual to a system that ranks BGCs according to response or non-response to the therapy.

Embodiment 18: The method of Embodiment 17, wherein when the one or more BGCs from microbes of the individual correlate to BGCs from the system associated with a response to the therapy, the individual is administered an effective amount of the therapy.

Embodiment 19: The method of Embodiment 17, wherein when the one or more BGCs from microbes of the individual correlate to BGCs from the system associated with non-response to the therapy, the individual is not administered the therapy.

Embodiment 20: The method of Embodiment 17, wherein the system is produced by analyzing BGCs from a plurality of individuals having received the therapy and that were responders or non-responders, followed by ranking of the BGCs according to statistical association to a responder phenotype.

Embodiment 21: The method of Embodiment 20, wherein production of the system comprises: (a) sequencing microbial gut DNA from the plurality of individuals to produce sequencing reads; (b) aligning sequencing reads into contigs of no less than 20K base pairs; (c) identifying the BGCs based on their sequence and grouping BGCs of similar sequence; (d) denoting BGCs as being from responders or from non-responders; and (e) ranking statistically the BGCs from responders.

Embodiment 22: The method of Embodiments 18 or 19, further comprising the step of administering an additional cancer therapy.

Embodiment 23: A method of treating an individual in need thereof, comprising the step of administering a therapeutically effective amount of a therapy to the individual that has one or more BGCs from gut microbes that are indicative of response to the therapy.

Embodiment 24: The method of Embodiment 23, further comprising comparing the sequence of one or more BGCs from gut microbes from the individual to the sequence of one or more BGCs from gut microbes from a plurality of individuals each having a known response or known non-response to the therapy.

Embodiment 25: The method of Embodiment 24, wherein treatment is administered to the individual when the sequence of one or more BGCs from gut microbes from the individual correlates to sequence of one or more BGCs from gut microbes from individuals having a response to the therapy.

Embodiment 26: The method of any one of Embodiments 23-25, wherein the therapy comprises one or more immune checkpoint immunotherapies.

Embodiment 27: A method of developing a therapy, comprising the steps of: identifying one or more metabolites produced from one or more BGCs from gut microbes from one or more individuals, wherein the BGCs are associated with a responder phenotype to a therapy; and testing the one or more metabolites for efficacy as the therapy.

Embodiment 28: The method of Embodiment 27, wherein the testing is in vitro, ex vivo, or in vivo.

Embodiment 29: The method of Embodiments 27 or 28, wherein the testing is as an immune checkpoint inhibitor.

Embodiment 30: The method of any one of Embodiments 27-29, wherein the testing is for activity against PD1, PD-L1, and/or CTLA-4.

Embodiment 31: The method of Embodiments 27 or 28, wherein the one or more metabolites are further modified.

Embodiment 32: The method of Embodiment 29, wherein the further modifications comprise alteration of one or more R groups on the one or more metabolites.

Embodiment 33: A method for training an artificial intelligence (AI) model for assessing or predicting clinical outcomes in immunotherapy patients using identified microbial biosynthetic genetic cluster (BGCs), the method comprising: receiving a first dataset comprising a biological sample from each patient in a first training cohort, wherein each patient in the first training cohort is subject to a common immunotherapy; generating a plurality of BGC clusters based on analysis of the biological samples from the patient cohort, each BGC cluster grouped by common homology; scoring each BGC cluster based on response to the immunotherapy; training the AI model using the scored BGC clusters, wherein the training comprises: identifying features in the scored BGC clusters relevant to immunotherapy response, and classifying the identified features based on their relative association to immunotherapy response; and validating the trained AI model using a second dataset comprising a biological sample from a patient in a second training cohort.

Embodiment 34: The method of Embodiment 33, wherein the AI model is a machine learning model.

Embodiment 35: The method of Embodiments 33 or 34, wherein the immunotherapy is a checkpoint therapy.

Embodiment 36: The method of any one of Embodiments 33 to 35, wherein the biological sample comprises a stool sample or a plasma sample.

Embodiment 37: The method of any one of Embodiments 33 to 36, wherein the classifying comprises ranking the identified features based on their relative association to immunotherapy response.

Embodiment 38: The method of any one of Embodiments 33 to 37, wherein the classifying comprises using a neural network, natural language processing, or a combination thereof.

Embodiment 39: A method for assessing or predicting clinical outcomes in immunotherapy patients using identified microbial biosynthetic genetic cluster (BGCs), the method comprising: receiving a dataset comprising a biological sample from a patient subject to a given immunotherapy; analyzing the dataset using an artificial intelligence (AI) model, wherein the AI model is trained using identified features in BGC clusters associated with a biological sample from each test patient in a training cohort, wherein each test patient in the training cohort is classified according to a known response to the given immunotherapy, and wherein the identified features are classified based on their relative association to immunotherapy response; identifying one or more features from the dataset common to the identified features from the trained AI model, and predicting the patient response to the immunotherapy by comparing the identified features from the patient dataset to the classified features from the trained AI model.

Embodiment 40: The method of Embodiment 39, wherein the AI model is a machine learning model.

Embodiment 41: The method of Embodiments 39 or 40, wherein the immunotherapy is a checkpoint therapy.

Embodiment 42: The method of any one of Embodiments 39 to 41, wherein the biological sample comprises a stool sample or a plasma sample.

Embodiment 43: The method of any one of Embodiments 39 to 42, wherein the identified features are ranked based on their relative association to immunotherapy response.

Embodiment 44: The method of any one of Embodiments 39 to 43, wherein the identified features are ranked using a neural network, natural language processing, or a combination thereof.

Embodiment 45: A method for identifying microbial biosynthetic genetic cluster (BGCs) related to a response to a therapy, the method comprising: obtaining genetic information of samples from a plurality of test subjects, wherein the plurality of test subjects include responders and non-responders, wherein the responders had a disease and were responsive to a therapy for the disease, and wherein the non-responders had the disease and were not responsive to the therapy for the disease; obtaining respective response information of the plurality of test subjects regarding each test subject's response to the therapy; categorizing the test subjects as responders and non-responders based on obtained response information according to an imaging-based tumor-specific response criterion; identifying microbial BGCs and respective genetic features using the genetic information, wherein each microbial BGC is categorized as a responsive BGC or a non-responsive BGC using the respective response information; grouping the microbial BGCs into cliques, wherein each clique has a subset of microbial BGCs having a genetic feature similarity score meeting or exceeding a similarity threshold, and where each clique is assigned to a clique response score based on a percentage of responsive BGCs; identifying target cliques from the cliques by having a clique response score meeting or exceeding a pre-set clique response score; and identifying target microbial BGCs from the target cliques based on the target microbial BGCs's correlation to a response to the therapy, where the correlation is determined using the obtained response information.

Embodiment 46: The method of Embodiment 45, wherein the disease is an immunological disease.

Embodiment 47: The method of Embodiment 46, wherein the immunological disease is a cancer or an autoimmune disease.

Embodiment 48: The method of Embodiment 47, wherein the cancer is non-small-cell-long cancer.

Embodiment 49: The method of any one or Embodiments 45 to 48, wherein the therapy is immune checkpoint inhibition therapy.

Embodiment 50: The method of Embodiment 49, wherein the immune checkpoint inhibition therapy comprises an inhibitor that inhibits cytotoxic T-lymphocyte-associated antigen 4 (CTLA-4), programmed cell death protein 1 (PD-1), or PDL-1.

Embodiment 51: The method of any one of Embodiments 45 to 50, wherein the samples are stool samples.

Embodiment 52: The method of Embodiment 51, wherein obtaining genetic information comprises obtaining stool samples from the test subjects.

Embodiment 53: The method of Embodiment 52, wherein obtaining genetic information comprises extracting microbial DNA from the stool samples.

Embodiment 54: The method of Embodiment 53, wherein obtaining genetic information comprises sequencing the microbial DNA using whole genome shotgun (WGS) sequencing.

Embodiment 55: The method of any one of Embodiments 45 to 54, wherein obtaining the genetic information comprises obtaining contigs of microbial genomics sequences.

Embodiment 56: The method of any one of Embodiments 45 to 55, wherein grouping the microbial BGCs into cliques comprises topological data analysis.

Embodiment 57: The method of Embodiment 56, wherein the topological data analysis comprises using direct graph-based network, topological data analysis network, or a combination thereof.

Embodiment 58: The method of any one of Embodiments 45 to 57, further comprising ranking the target microbial BGCs based on their correlation with the lest subjects' response to the therapy.

Embodiment 59: The method of Embodiment 58, wherein ranking the target microbial BGCs comprises using neural network, natural language processing, or a combination thereof.

Embodiment 60: The method of any one of Embodiments 45 to 59, further comprising testing the target microbial BGCs in vitro to obtain immunologic and metabolomic information of the target microbial BGCs.

Embodiment 61: The method of Embodiment 60, wherein testing the target microbial BGCs in vitro comprises: reducing expression of the target microbial BGCs in bacteria; and screening for a change in levels of metabolite secreted from the bacteria after reducing expression to obtain the metabolomic information of the target microbial BGCs.

Embodiment 62: The method of Embodiments 60 or 61, wherein testing the target microbial BGCs in vitro comprises: reducing expression of the target microbial BGCs in bacteria; incubated human cells with a lysate obtained from the bacteria after reducing expression; and screening for a change of cytokine levels of the human cells after incubation to obtain the immunologic information of the target microbial BGCs.

Embodiment 63: The method of any one of Embodiments 60 to 62, wherein the human cells comprise peripheral blood mononuclear cell (PBMC).

Embodiment 64: The method of any one of Embodiments 60 to 63, further comprising: grouping the target microbial BGCs into refined cliques, wherein each refined clique shares similar immunologic and metabolomic information meeting or exceeding a second similarity threshold; and ranking refined cliques according to correlation between target microbial BGCs of corresponding refined cliques and respective immunologic and metabolomic information of the target microbial BGCs.

Embodiment 65: A non-transitory computer-readable medium storing computer instructions that, when executed by a computer, cause the computer to perform a method for identifying microbial biosynthetic genetic cluster (BGCs) related to a response to a therapy, the method comprising: obtaining genetic information of samples from a plurality of test subjects, wherein the plurality of test subjects include responders and non-responders, wherein the responders had a disease and were responsive to a therapy for the disease, and wherein the non-responders had the disease and were not responsive to the therapy for the disease; obtaining respective response information of the plurality of test subjects regarding each test subject's response to the therapy; categorizing the test subjects as responders and non-responders based on obtained response information according to an imaging-based tumor-specific response criterion; identifying microbial BGCs and respective genetic features using the genetic information, wherein each microbial BGC is categorized as a responsive BGC or a non-responsive BGC using the respective response information; grouping the microbial BGCs into cliques, wherein each clique has a subset of microbial BGCs having a genetic feature similarity score meeting or exceeding a similarity threshold, and where each clique is assigned to a clique response score based on a percentage of responsive BGCs; identifying target cliques from the cliques by having a clique response score meeting or exceeding a pre-set clique response score; and identifying target microbial BGCs from the target cliques based on the target microbial BGCs's correlation to a response to the therapy, where the correlation is determined using the obtained response information.

Embodiment 66: The non-transitory computer-readable medium of Embodiment 65, wherein grouping the microbial BGCs into cliques comprises topological data analysis.

Embodiment 67: The non-transitory computer-readable medium of Embodiment 66, wherein the topological data analysis comprises using direct graph-based network, topological data analysis network, or a combination thereof.

Embodiment 68: The non-transitory computer-readable medium of any one of Embodiments 65 to 68, wherein the method further comprises ranking the target microbial BGCs based on their correlation with the test subjects' response to the therapy.

Embodiment 69: The non-transitory computer-readable medium of Embodiment 68, wherein ranking BGCs comprises using neural network, natural language processing, or a combination thereof.

Embodiment 70: The non-transitory computer-readable medium of any one of Embodiments 65 to 69, wherein the method further comprises testing the target microbial BGCs in vitro to obtain immunologic and metabolomic information of the target microbial BGCs.

Embodiment 71: The non-transitory computer-readable medium of Embodiment 70, wherein testing the target microbial BGCs in vitro comprises: reducing expression of the target microbial BGCs in bacteria; and screening for a change of metabolite levels secreted from the bacteria after reducing expression to obtain the metabolomic information of the target microbial BGCs.

Embodiment 72: The non-transitory computer-readable medium of Embodiment 71, wherein testing the target microbial BGCs in vitro comprises: reducing expression of the target microbial BGCs in bacteria; incubated human cells with a lysate from the bacteria after reducing expression; and screening for a change of cytokine levels of the human cells after incubation to obtain the immunologic information of the target microbial BGCs.

Embodiment 73: The non-transitory computer-readable medium of Embodiments 71 or 72, wherein the human cells comprise peripheral blood mononuclear cell (PBMC).

Embodiment 74: The non-transitory computer-readable medium of any one of Embodiments 71 to 73, wherein the method further comprises: grouping the target microbial BGCs into refined cliques, wherein each refined clique shares similar immunologic and metabolomic information meeting or exceeding a second similarity threshold; and ranking refined cliques according to correlation between target microbial BGCs of corresponding refined cliques and respective immunologic and metabolomic information of the target microbial BGCs.

Embodiment 75: A system for identifying microbial biosynthetic genetic cluster (BGCs) related to a response to a therapy, comprising: a data store configured to store a data set storing genetic information of samples from a plurality of test subjects and respective response information of the plurality of test subjects regarding each test subject's response to the therapy, wherein the plurality of test subjects include responders and non-responders, wherein the responders had a disease and were responsive to a therapy for the disease, and wherein the non-responders had the disease and were not responsive to the therapy for the disease; and a computing device communicatively connected to the data store and configured to receive the data set, the computing device comprising a BGC analysis engine configured to categorize the test subjects as responders and non-responders based on obtained response information according to an imaging-based tumor-specific response criterion; identify microbial BGCs and respective genetic features using the genetic information, wherein each microbial BGC is categorized as a responsive BGC or a non-responsive BGC using the respective response information; group the microbial BGCs into cliques, wherein each clique has a subset of microbial BGCs having a genetic feature similarity score meeting or exceeding a similarity threshold, and where each clique is assigned to a clique response score based on a percentage of responsive BGCs; identify target cliques from the cliques by having a clique response score meeting or exceeding a pre-set clique response score; and identify target microbial BGCs from the target cliques based on the target microbial BGCs's correlation to a response to the therapy meeting or exceeding a pre-set correlation criterion, where the correlation is determined using the obtained response information.

Embodiment 76: The system of Embodiment 75, wherein grouping the microbial BGCs into cliques comprises topological data analysis.

Embodiment 77: The system of Embodiment 76, wherein the topological data analysis comprises using direct graph-based network, topological data analysis network, or a combination thereof.

Embodiment 78: The system of any one of Embodiments 75 to 77, further comprising ranking the target microbial BGCs based on their correlation with the lest subjects' response to the therapy.

Embodiment 79: The system of Embodiment 78, wherein ranking BGCs comprises using neural network, natural language processing, or a combination thereof.

Embodiment 80: The system of any one of Embodiments 75 to 79, wherein the BGC analysis engine is further configured to test the target microbial BGCs in vitro to obtain immunologic and metabolomic information of the target microbial BGCs.

Embodiment 81: The system of Embodiment 80, wherein testing the target microbial BGCs in vitro comprises: reducing expression of the target microbial BGCs in bacteria; and screening for a change of metabolite levels secreted from the bacteria to obtain the metabolomic information of the target microbial BGCs.

Embodiment 82: The system of Embodiments 80 or 81, wherein testing the target microbial BGCs in vitro comprises: reducing expression of the target microbial BGCs in bacteria; incubated human cells with a lysate from the bacteria after reducing expression; and screening for a change of cytokine levels of the human cells after incubation to obtain the immunologic information of the target microbial BGCs.

Embodiment 83: The system of any one of Embodiments 80 to 82, wherein the human cells comprise peripheral blood mononuclear cell (PBMC).

Embodiment 84: The system of any one of Embodiments 80 to 83, wherein the BGC analysis engine is further configured to: group the target microbial BGCs into refined cliques, wherein each refined clique shares similar immunologic and metabolomic information meeting or exceeding a second similarity threshold; and rank refined cliques according to correlation between target microbial BGCs in corresponding refined cliques and respective immunologic and metabolomic information of the target microbial BGCs.

Embodiment 85: A method for identifying microbial biosynthetic genetic cluster (BGCs) related to a response to a therapy, comprising: obtaining genetic information of samples from a plurality of test subjects, wherein the plurality of test subjects include responders and non-responders, w herein the responders had a disease and were responsive to a therapy for the disease, and wherein the non-responders had the disease and were not responsive to the therapy for the disease; obtaining respective response information of the plurality of test subjects regarding each test subject's response to the therapy; categorizing the test subjects as responders and non-responders based on obtained response information; identifying microbial BGCs and respective genetic features using the genetic information, wherein each microbial BGC is categorized as responsive BGC or non-responsive BGC using the respective response information; grouping the microbial BGCs into cliques in a topological graph, wherein each clique has a subset of microbial BGCs having a genetic feature similarity score meeting or exceeding a similarity threshold, and where each clique is assigned to a clique response score based on a percentage of responsive BGCs, and identifying target cliques from the cliques according to correlation of each clique to a response to the therapy using the respective response information, wherein the target cliques are used for identifying microbial biosynthetic genetic cluster (BGCs) related to a response to a therapy from the target cliques.

Embodiment 86: A method for ranking microbial biosynthetic genetic cluster (BGCs) related to a response to a therapy, comprising: obtaining genetic information of samples from a plurality of test subjects, wherein the plurality of test subjects include responders and non-responders, wherein the responders had a disease and were responsive to a therapy for the disease, and wherein the non-responders had the disease and were not responsive to the therapy for the disease; obtaining respective response information of the plurality of test subjects regarding each test subject's response to the therapy; categorizing the test subjects as responders and non-responders based on obtained response information; identifying microbial BGCs and respective genetic features using the genetic information, wherein each microbial BGC is categorized as a responsive BGC or a non-responsive BGC using the respective response information; grouping the microbial BGCs into cliques, wherein each clique has a subset of microbial BGCs having a genetic feature similarity score meeting or exceeding a similarity threshold; and ranking the cliques with a neural network built using the respective response information, wherein a clique's higher ranking indicates the clique's higher correlation to a response to the therapy; and ranking microbial BGCs in cliques with a ranking meeting or exceeding a pre-set ranking threshold using the respective response information.

Embodiment 87: A non-transitory computer-readable medium storing computer instructions that, when executed by a computer, cause the computer to perform a method for training an artificial intelligence (AI) model for assessing or predicting clinical outcomes in immunotherapy patients using identified microbial biosynthetic genetic cluster (BGCs), the method comprising: receiving a first dataset comprising a biological sample from each patient in a first training cohort, wherein each patient in the first training cohort is subject to a common immunotherapy; generating a plurality of BGC clusters based on analysis of the biological samples from the patient cohort, each BGC cluster grouped by common homology; scoring each BGC cluster based on response to the immunotherapy; training the AI model using the scored BGC clusters, wherein the training comprises: identifying features in the scored BGC clusters relevant to immunotherapy response, and classifying the identified features based on their relative association to immunotherapy response); and validating the trained AI model using a second dataset comprising a biological sample from a patient in a second training cohort.

Embodiment 88: A non-transitory computer-readable medium storing computer instructions that, when executed by a computer, cause the computer to perform a method for assessing or predicting clinical outcomes in immunotherapy patients using identified microbial biosynthetic genetic cluster (BGCs), the method comprising: receiving a dataset comprising a biological sample from a patient subject to a given immunotherapy; analyzing the dataset using an artificial intelligence (AI) model, wherein the AI model is trained using identified features in BGC clusters associated with a biological sample from each test patient in a training cohort, wherein each test patient in the training cohort is classified according to a known response to the given immunotherapy, and wherein the identified features are classified based on their relative association to immunotherapy response; identifying one or more features from the dataset common to the identified features from the trained AI model, and predicting the patient response to the immunotherapy by comparing the identified features from the patient dataset to the classified features from the trained AI model.

What is claimed is:

1. A method of identifying microbial biosynthetic genetic cluster (BGCs) related to a response to a therapy in an individual, the method comprising:
(a) sequencing microbial DNA from samples from a plurality of individuals having received a therapy, wherein a first group of individuals are responders to the therapy and a second group of individuals are non-responders to the therapy;
(b) collating sequencing information from each of the plurality of individuals to produce contiguous sequencing reads (contigs);
(c) identifying one or more BGCs from the respective contigs;
(d) assigning BGCs as originating from the first group of individuals or the second group of individuals; and
(e) ranking the BGCs according to statistical association to a responder phenotype.

2. The method of claim 1, wherein the microbial DNA is bacterial DNA.

3. The method of claim 1 or 2, wherein the sample is from the gut, from blood, or a mixture thereof.

4. The method of any one of the preceding claims, wherein the sample is from the stool.

5. The method of any one of the preceding claims, wherein DNA of the individuals in the plurality is not sequenced or not intended to be sequenced.

6. The method of any one of the preceding claims, wherein the sequencing is by whole genome shotgun sequencing.

7. The method of any one of the preceding claims, wherein at least the majority of the contigs are no less than about 20K base pairs in length.

8. The method of claim 1 or 2, wherein the therapy is cancer therapy.

9. The method of claim 3, wherein the cancer therapy is immunotherapy.

10. The method of claim 4, wherein the immunotherapy is immune checkpoint immunotherapy.

11. The method of claim 5, wherein the immune checkpoint immunotherapy is anti-programmed cell death protein 1 (PD1) therapy, anti-Programmed death-ligand 1 (PD-L1) therapy, anti-cytotoxic T-lymphocyte-associated antigen 4 (CTLA-4) therapy, or a combination thereof.

12. The method of claim 1, wherein the responders are complete responders or partial responders.

13. The method of any one of the preceding claims, further comprising the step of comparing one or more BGC sequences from a gut microbe from an individual having an unknown response to the therapy to the ranked BGCs, thereby determining an indication of a response to the therapy for the individual.

14. The method of claim 13, wherein when the individual is considered to have one or more BGC sequences associated with a responder phenotype, the individual is given the therapy.

15. The method of claim 13, wherein when the individual is considered not to have one or more BGC sequences associated with a responder phenotype, the individual is not given the therapy.

16. The method of claim 15, wherein the individual is administered a therapeutically effective amount of one or more therapies that are not immune checkpoint immunotherapies.

17. A method of determining a treatment regimen for an individual in need of a therapy, comprising the step of comparing the sequence of one or more BGCs from gut microbes of the individual to a system that ranks BGCs according to response or non-response to the therapy.

18. The method of claim 17, wherein when the one or more BGCs from microbes of the individual correlate to BGCs from the system associated with a response to the therapy, the individual is administered an effective amount of the therapy.

19. The method of claim 17, wherein when the one or more BGCs from microbes of the individual correlate to BGCs from the system associated with non-response to the therapy, the individual is not administered the therapy.

20. The method of claim 17, wherein the system is produced by analyzing BGCs from a plurality of individuals having received the therapy and that were responders or non-responders, followed by ranking of the BGCs according to statistical association to a responder phenotype.

21. The method of claim 20, wherein production of the system comprises:
(a) sequencing microbial gut DNA from the plurality of individuals to produce sequencing reads;
(b) aligning sequencing reads into contigs of no less than 20K base pairs;
(c) identifying the BGCs based on their sequence and grouping BGCs of similar sequence;
(d) denoting BGCs as being from responders or from non-responders; and
(e) ranking statistically the BGCs from responders.

22. The method of claim 18 or 19, further comprising the step of administering an additional cancer therapy.

23. A method of treating an individual in need thereof, comprising the step of administering a therapeutically effective amount of a therapy to the individual that has one or more BGCs from gut microbes that are indicative of response to the therapy.

24. The method of claim 23, further comprising comparing the sequence of one or more BGCs from gut microbes from the individual to the sequence of one or more BGCs from gut microbes from a plurality of individuals each having a known response or known non-response to the therapy.

25. The method of claim 24, wherein treatment is administered to the individual when the sequence of one or more BGCs from gut microbes from the individual correlates to sequence of one or more BGCs from gut microbes from individuals having a response to the therapy.

26. The method of any one of claims 23-25, wherein the therapy comprises one or more immune checkpoint immunotherapies.

27. A method of developing a therapy, comprising the steps of:
identifying one or more metabolites produced from one or more BGCs from gut microbes from one or more individuals, wherein the BGCs are associated with a responder phenotype to a therapy; and
testing the one or more metabolites for efficacy as the therapy.

28. The method of claim 27, wherein the testing is in vitro, ex vivo, or in vivo.

29. The method of claim 27 or 28, wherein the testing is as an immune checkpoint inhibitor.

30. The method of any one of claims 27-29, wherein the testing is for activity against PD1, PD-L1, and/or CTLA-4.

31. The method of claim 27 or 28, wherein the one or more metabolites are further modified.

32. The method of claim 29, wherein the further modifications comprise alteration of one or more R groups on the one or more metabolites.

33. A method for training an artificial intelligence (AI) model for assessing or predicting clinical outcomes in immunotherapy patients using identified microbial biosynthetic genetic cluster (BGCs), the method comprising:
receiving a first dataset comprising a biological sample from each patient in a first training cohort, wherein each patient in the first training cohort is subject to a common immunotherapy;
generating a plurality of BGC clusters based on analysis of the biological samples from the patient cohort, each BGC cluster grouped by common homology;
scoring each BGC cluster based on response to the immunotherapy;
training the AI model using the scored BGC clusters, wherein the training comprises:
identifying features in the scored BGC clusters relevant to immunotherapy response, and
classifying the identified features based on their relative association to immunotherapy response;
and
validating the trained AI model using a second dataset comprising a biological sample from a patient in a second training cohort.

34. A method for assessing or predicting clinical outcomes in immunotherapy patients using identified microbial biosynthetic genetic cluster (BGCs), the method comprising:
receiving a dataset comprising a biological sample from a patient subject to a given immunotherapy;
analyzing the dataset using an artificial intelligence (AI) model,
wherein the AI model is trained using identified features in BGC clusters associated with a biological sample from each test patient in a training cohort,
wherein each test patient in the training cohort is classified according to a known response to the given immunotherapy, and
wherein the identified features are classified based on their relative association to immunotherapy response;
identifying one or more features from the dataset common to the identified features from the trained AI model, and
predicting the patient response to the immunotherapy by comparing the identified features from the patient dataset to the classified features from the trained AI model.

35. A method for identifying microbial biosynthetic genetic cluster (BGCs) related to a response to a therapy, the method comprising:
obtaining genetic information of samples from a plurality of test subjects, wherein the plurality of test subjects include responders and non-responders, wherein the responders had a disease and were responsive to a therapy for the disease, and wherein the non-responders had the disease and were not responsive to the therapy for the disease;
obtaining respective response information of the plurality of test subjects regarding each test subject's response to the therapy;
categorizing the test subjects as responders and non-responders based on obtained response information according to an imaging-based tumor-specific response criterion;
identifying microbial BGCs and respective genetic features using the genetic information, wherein each microbial BGC is categorized as a responsive BGC or a non-responsive BGC using the respective response information;
grouping the microbial BGCs into cliques, wherein each clique has a subset of microbial BGCs having a genetic feature similarity score meeting or exceeding a similarity threshold, and where each clique is assigned to a clique response score based on a percentage of responsive BGCs;
identifying target cliques from the cliques by having a clique response score meeting or exceeding a pre-set clique response score; and
identifying target microbial BGCs from the target cliques based on the target microbial BGCs's correlation to a response to the therapy, where the correlation is determined using the obtained response information.

36. The method of claim 35, wherein the disease is an immunological disease.

37. The method of claim 36, wherein the immunological disease is a cancer or an autoimmune disease.

38. The method of claim 37, wherein the cancer is non-small-cell-long cancer.

39. The method of claim 35, wherein the therapy is immune checkpoint inhibition therapy.

40. The method of claim 39, wherein the immune checkpoint inhibition therapy comprises an inhibitor that inhibits cytotoxic T-lymphocyte-associated antigen 4 (CTLA-4), programmed cell death protein 1 (PD-1), or PDL-1.

41. The method of claim 35, wherein the samples are stool samples.

42. The method of claim 41, wherein obtaining genetic information comprises obtaining stool samples from the test subjects.

43. The method of claim 42, wherein obtaining genetic information comprises extracting microbial DNA from the stool samples.

44. The method of claim 43, wherein obtaining genetic information comprises sequencing the microbial DNA using whole genome shotgun (WGS) sequencing.

45. The method of claim 35, wherein obtaining the genetic information comprises obtaining contigs of microbial genomics sequences.

46. The method of claim 35, wherein grouping the microbial BGCs into cliques comprises topological data analysis.

47. The method of claim 46, wherein the topological data analysis comprises using direct graph-based network, topological data analysis network, or a combination thereof.

48. The method of claim 35, further comprising ranking the target microbial BGCs based on their correlation with the lest subjects' response to the therapy.

49. The method of claim 48, wherein ranking the target microbial BGCs comprises using neural network, natural language processing, or a combination thereof.

50. The method of claim 35, further comprising testing the target microbial BGCs in vitro to obtain immunologic and metabolomic information of the target microbial BGCs.

51. The method of claim 50, wherein testing the target microbial BGCs in vitro comprises:
reducing expression of the target microbial BGCs in bacteria; and
screening for a change in levels of metabolite secreted from the bacteria after reducing expression to obtain the metabolomic information of the target microbial BGCs.

52. The method of claim 50, wherein testing the target microbial BGCs in vitro comprises:
reducing expression of the target microbial BGCs in bacteria;
incubated human cells with a lysate obtained from the bacteria after reducing expression; and
screening for a change of cytokine levels of the human cells after incubation to obtain the immunologic information of the target microbial BGCs.

53. The method of claim 50, wherein the human cells comprise peripheral blood mononuclear cell (PBMC).

54. The method of claim 50, further comprising:
grouping the target microbial BGCs into refined cliques, wherein each refined clique shares similar immunologic and metabolomic information meeting or exceeding a second similarity threshold; and
ranking refined cliques according to correlation between target microbial BGCs of corresponding refined cliques and respective immunologic and metabolomic information of the target microbial BGCs.

55. A non-transitory computer-readable medium storing computer instructions that, when executed by a computer, cause the computer to perform a method for identifying microbial biosynthetic genetic cluster (BGCs) related to a response to a therapy, the method comprising:
obtaining genetic information of samples from a plurality of test subjects, wherein the plurality of test subjects include responders and non-responders, wherein the responders had a disease and were responsive to a therapy for the disease, and wherein the non-responders had the disease and were not responsive to the therapy for the disease;
obtaining respective response information of the plurality of test subjects regarding each test subject's response to the therapy;
categorizing the test subjects as responders and non-responders based on obtained response information according to an imaging-based tumor-specific response criterion;
identifying microbial BGCs and respective genetic features using the genetic information, wherein each microbial BGC is categorized as a responsive BGC or a non-responsive BGC using the respective response information;
grouping the microbial BGCs into cliques, wherein each clique has a subset of microbial BGCs having a genetic feature similarity score meeting or exceeding a similarity threshold, and where each clique is assigned to a clique response score based on a percentage of responsive BGCs;
identifying target cliques from the cliques by having a clique response score meeting or exceeding a pre-set clique response score; and
identifying target microbial BGCs from the target cliques based on the target microbial BGCs's correlation to a response to the therapy, where the correlation is determined using the obtained response information.

56. The non-transitory computer-readable medium of claim 55, wherein grouping the microbial BGCs into cliques comprises topological data analysis.

57. The non-transitory computer-readable medium of claim 56, wherein the topological data analysis comprises using direct graph-based network, topological data analysis network, or a combination thereof.

58. The non-transitory computer-readable medium of claim 55, wherein the method further comprises ranking the target microbial BGCs based on their correlation with the test subjects' response to the therapy.

59. The non-transitory computer-readable medium of claim 58, wherein ranking BGCs comprises using neural network, natural language processing, or a combination thereof.

60. The non-transitory computer-readable medium of claim 55, wherein the method further comprises testing the target microbial BGCs in vitro to obtain immunologic and metabolomic information of the target microbial BGCs.

61. The non-transitory computer-readable medium of claim 60, wherein testing the target microbial BGCs in vitro comprises:
reducing expression of the target microbial BGCs in bacteria; and
screening for a change of metabolite levels secreted from the bacteria after reducing expression to obtain the metabolomic information of the target microbial BGCs.

62. The non-transitory computer-readable medium of claim 61, wherein testing the target microbial BGCs in vitro comprises:
reducing expression of the target microbial BGCs in bacteria;
incubated human cells with a lysate from the bacteria after reducing expression; and
screening for a change of cytokine levels of the human cells after incubation to obtain the immunologic information of the target microbial BGCs.

63. The non-transitory computer-readable medium of claim 61, wherein the human cells comprise peripheral blood mononuclear cell (PBMC).

64. The non-transitory computer-readable medium of claim 61, wherein the method further comprises:
grouping the target microbial BGCs into refined cliques, wherein each refined clique shares similar immunologic and metabolomic information meeting or exceeding a second similarity threshold; and
ranking refined cliques according to correlation between target microbial BGCs of corresponding refined cliques and respective immunologic and metabolomic information of the target microbial BGCs.

65. A system for identifying microbial biosynthetic genetic cluster (BGCs) related to a response to a therapy, comprising:
a data store configured to store a data set storing genetic information of samples from a plurality of test subjects and respective response information of the plurality of test subjects regarding each test subject's response to the therapy, wherein the plurality of test subjects include responders and non-responders, wherein the responders had a disease and were responsive to a therapy for the disease, and wherein the non-responders had the disease and were not responsive to the therapy for the disease; and
a computing device communicatively connected to the data store and configured to receive the data set, the computing device comprising a BGC analysis engine configured to
categorize the test subjects as responders and non-responders based on obtained response information according to an imaging-based tumor-specific response criterion;
identify microbial BGCs and respective genetic features using the genetic information, wherein each microbial BGC is categorized as a responsive BGC or a non-responsive BGC using the respective response information;
group the microbial BGCs into cliques, wherein each clique has a subset of microbial BGCs having a genetic feature similarity score meeting or exceeding a similarity threshold, and where each clique is assigned to a clique response score based on a percentage of responsive BGCs;
identify target cliques from the cliques by having a clique response score meeting or exceeding a pre-set clique response score; and
identify target microbial BGCs from the target cliques based on the target microbial BGCs's correlation to a response to the therapy meeting or exceeding a pre-set correlation criterion, where the correlation is determined using the obtained response information.

66. The system of claim 65, wherein grouping the microbial BGCs into cliques comprises topological data analysis.

67. The system of claim 66, wherein the topological data analysis comprises using direct graph-based network, topological data analysis network, or a combination thereof.

68. The system of claim 65, further comprising ranking the target microbial BGCs based on their correlation with the lest subjects' response to the therapy.

69. The system of claim 68, wherein ranking BGCs comprises using neural network, natural language processing, or a combination thereof.

70. The system of claim 65, wherein the BGC analysis engine is further configured to test the target microbial BGCs in vitro to obtain immunologic and metabolomic information of the target microbial BGCs.

71. The system of claim 70, wherein testing the target microbial BGCs in vitro comprises:
reducing expression of the target microbial BGCs in bacteria; and
screening for a change of metabolite levels secreted from the bacteria to obtain the metabolomic information of the target microbial BGCs.

72. The system of claim 70, wherein testing the target microbial BGCs in vitro comprises:
reducing expression of the target microbial BGCs in bacteria;
incubated human cells with a lysate from the bacteria after reducing expression; and
screening for a change of cytokine levels of the human cells after incubation to obtain the immunologic information of the target microbial BGCs.

73. The system of claim 70, wherein the human cells comprise peripheral blood mononuclear cell (PBMC).

74. The system of claim 70, wherein the BGC analysis engine is further configured to:
group the target microbial BGCs into refined cliques, wherein each refined clique shares similar immunologic and metabolomic information meeting or exceeding a second similarity threshold; and
rank refined cliques according to correlation between target microbial BGCs in corresponding refined cliques and respective immunologic and metabolomic information of the target microbial BGCs.

75. A method for identifying microbial biosynthetic genetic cluster (BGCs) related to a response to a therapy, comprising:
obtaining genetic information of samples from a plurality of test subjects, wherein the plurality of test subjects include responders and non-responders, w herein the responders had a disease and were responsive to a therapy for the disease, and wherein the non-responders had the disease and were not responsive to the therapy for the disease;
obtaining respective response information of the plurality of test subjects regarding each test subject's response to the therapy;
categorizing the test subjects as responders and non-responders based on obtained response information;
identifying microbial BGCs and respective genetic features using the genetic information, wherein each microbial BGC is categorized as responsive BGC or non-responsive BGC using the respective response information;
grouping the microbial BGCs into cliques in a topological graph, wherein each clique has a subset of microbial BGCs having a genetic feature similarity score meeting or exceeding a similarity threshold, and where each clique is assigned to a clique response score based on a percentage of responsive BGCs
identifying target cliques from the cliques according to correlation of each clique to a response to the therapy using the respective response information, wherein the target cliques are used for identifying microbial biosynthetic genetic cluster (BGCs) related to a response to a therapy from the target cliques.

76. A method for ranking microbial biosynthetic genetic cluster (BGCs) related to a response to a therapy, comprising:
obtaining genetic information of samples from a plurality of test subjects, wherein the plurality of test subjects include responders and non-responders, wherein the responders had a disease and were responsive to a therapy for the disease, and wherein the non-responders had the disease and were not responsive to the therapy for the disease;
obtaining respective response information of the plurality of test subjects regarding each test subject's response to the therapy;
categorizing the test subjects as responders and non-responders based on obtained response information;
identifying microbial BGCs and respective genetic features using the genetic information, wherein each microbial BGC is categorized as a responsive BGC or a non-responsive BGC using the respective response information;
grouping the microbial BGCs into cliques, wherein each clique has a subset of microbial BGCs having a genetic feature similarity score meeting or exceeding a similarity threshold; and
ranking the cliques with a neural network built using the respective response information, wherein a clique's higher ranking indicates the clique's higher correlation to a response to the therapy; and
ranking microbial BGCs in cliques with a ranking meeting or exceeding a pre-set ranking threshold using the respective response information.
